(19) United States
(12) Patent Application Publication
(10) Pub. No.: US 2008/0058783 A1
(43) Pub. Date: Mar. 6, 2008

(54) Handheld Photocosmetic Device

(75) Inventors: Gregory B. Altshuler, Lincoln, MA (US); Ilya Yaroslavsky, North Andover, MA (US); Stewart Wilson, Billerica, MA (US); James S. Cho, Westford, MA (US); Oldrich M. Laznicka JR., Wellesley, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Palomar Medical Technologies, Inc., Burlington, MA (US)

(21) Appl. No.: 11/769,604

(22) Filed: Jun. 27, 2007

Related U.S. Application Data
(63) Continuation-in-part of application No. 11/415,363, filed on May 1, 2006.
(63) Continuation-in-part of application No. 11/415,362, filed on May 1, 2006.
(63) Continuation-in-part of application No. 11/415,359, filed on May 1, 2006.
(63) Continuation-in-part of application No. 11/097,841, filed on Apr. 1, 2005.
(63) Continuation-in-part of application No. 11/098,000, filed on Apr. 1, 2005.
(63) Continuation-in-part of application No. 11/098,036, filed on Apr. 1, 2005.
(63) Continuation-in-part of application No. 11/098,015, filed on Apr. 1, 2005.
(63) Continuation-in-part of application No. 10/702,104, filed on Nov. 4, 2003.
(60) Provisional application No. 60/816,743, filed on Jun. 27, 2006.
(60) Provisional application No. 60/857,154, filed on Nov. 6, 2006.
(60) Provisional application No. 60/781,083, filed on Mar. 10, 2006.
(60) Provisional application No. 60/781,083, filed on Mar. 10, 2006.
(60) Provisional application No. 60/781,083, filed on Mar. 10, 2006.

(51) Int. Cl. A61B 18/18

(52) U.S. Cl. 606/9

(57) ABSTRACT

The present invention discloses handheld photocosmetic devices that can be utilized to apply EMR to the skin, e.g., to achieve fractional treatment of the skin. The invention discloses effective fractional photocosmetic devices for use in by a consumer in a non-medical and or non-professional setting. Thus, embodiments of such devices are disclosed herein that have one or more of the following attributes: capable of performing one or more cosmetic and/or dermatological treatments; efficacious for such treatments; durable; relatively inexpensive; relatively simple in design; smaller than existing professional devices (with some embodiments being completely self-contained and hand-held); safe for use by non-professionals; and/or not painful to use (or only mildly painful).

### RELATED APPLICATIONS

This application is a continuation-in-part application of U.S. application Ser. Nos. 11/097,841, 11/098,000, 11/098,036, and 11/098,015, each of which was filed Apr. 1, 2005 and entitled “Methods and products for producing lattices of EMR-treated islets in tissues, and uses therefore.” and each of which claims priority to U.S. Provisional Application No. 60/561,052, filed Apr. 9, 2004, U.S. Provisional Application No. 60/614,382, filed Sep. 29, 2004, U.S. Provisional Application No. 60/641,616, filed Jan. 5, 2005, and U.S. Provisional Application No. 60/620,734, filed Oct. 21, 2004; and each of which is also a continuation-in-part of U.S. patent application Ser. No. 10/080,652, filed Feb. 22, 2002, now abandoned, which claims priority to U.S. Provisional Application No. 60/272,745, filed Mar. 2, 2001.

This application also claims priority from U.S. application Ser. Nos. 11/415,363, 11/415,362, and 11/415,359, each of which was filed on May 1, 2006 and entitled “Photocosmetic Device”, each of which claims priority to U.S. Provisional Application 60/781,083, filed Mar. 10, 2006.

This application also claims priority from U.S. Provisional Application Ser. No. 60/816,743, filed Jun. 27, 2006, entitled “Handheld Photocosmetic Device” and U.S. Provisional Application Ser. No. 60/857,154, filed Nov. 6, 2007, entitled “Methods and Products for Producing Lattices of EMR-Treated Islets in Tissues, and Uses Therefore.”

Each of these applications to which this application claims priority are hereby incorporated by reference in their entirety.

### BACKGROUND OF THE INVENTION

1. Field of the Invention

The present invention relates generally to photocosmetic devices, and more particularly to handheld photocosmetic fractional devices that can be utilized, for example, by a consumer to apply electromagnetic radiation (“EMR”) to the skin to perform cosmetic and dermatological treatments.

2. Description of the Related Art

Electromagnetic radiation, particularly in the form of laser light or other optical radiation, has been used in a variety of cosmetic and medical applications, including uses in dermatology, dentistry, opthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues. As a general matter, the EMR treatment is typically designed to (a) deliver one or more particular wavelengths (or a particular continuous range of wavelengths) of EMR to a tissue to induce a particular chemical reaction, (b) deliver EMR energy to a tissue to cause an increase in temperature, or (c) deliver EMR energy to a tissue to damage or destroy cellular or extracellular structures, such as for skin remodeling.

For skin remodeling, absorption of optical energy by water is widely used in two approaches: ablative skin resurfacing, typically performed with either CO₂ (10.6 μm) or Er:YAG (2.94 μm) lasers, and non-ablative skin remodeling using a combination of deep skin heating with light from Nd:YAG (1.34 μm), Er:glass (1.56 μm) or diode laser (1.44 μm) and skin surface cooling for selective damage of sub-epidermal tissue. Nevertheless, in both cases, a healing response of the body is initiated as a result of the limited thermal damage, with the final outcome of new collagen formation and modification of the dermal collagen/elastin matrix. These changes manifest themselves in smoothing out rhytides and general improvement of skin appearance and texture (often referred to as “skin rejuvenation”).

The principal difference between the two techniques is the region of body where damage is initiated. In the resurfacing approach, the full thickness of the epidermis and a portion of upper dermis are ablated and/or coagulated. In the non-ablative approach, the zone of coagulation is shifted deeper into the tissue, with the epidermis being left intact. In practice, this is achieved by using different wavelengths: very shallow-penetrating ones in the ablative techniques (absorption coefficients of ˜900 cm⁻¹ and ˜13000 cm⁻¹ for CO₂ and Er:YAG wavelengths, respectively) and deeper-penetrating ones in the non-ablative techniques (absorption coefficients between 5 and 25 cm⁻¹). In addition, contact or spray cooling is applied to skin surface in non-ablative techniques, providing thermal protection for the epidermis. Resurfacing techniques have demonstrated significantly higher clinical efficacy. One drawback, which severely limited popularity of this treatment in the recent years, is a prolonged post-operative period requiring continuous care.

Non-ablative techniques offer considerably reduced risk of side effects and are much less demanding on post-operative care. However, clinical efficacy of the non-ablative procedure is often unsatisfactory. The reasons for such differences in the clinical outcomes of the two procedures are not completely understood. However, one possibility is that damage (or lack thereof) to the epidermis may be a factor determining both safety and efficacy outcomes. Destruction of the protective outer epidermal barrier (in particular, the stratum corneum) in the course of ablative skin resurfacing increases chances of wound contamination and potential complications. At the same time, release of growth factors (in particular, TGF-oe) by epidermal cells have been shown to play a crucial role in the wound healing process and, therefore, in the final skin remodeling. This process does not occur if the epidermis is intact.

In the cosmetic field for the treatment of various skin conditions, methods and devices have been developed that irradiate or cause damage in a portion of the tissue area and/or volume being treated. These methods and devices have become known as fractional technology. Fractional technology is thought to be a safer method of treatment of skin for cosmetic purposes, because the damage occurs within smaller sub-volumes or islets within the larger volume being treated. The tissue surrounding the islets is spared from the damage. Because the resulting islets are surrounded by neighboring healthy tissue the healing process is thorough and fast. Examples of devices that have been used to treat the skin during cosmetic procedures such as skin rejuvenation include the Palomar® LuxIR, which delivers infrared light to the surface of the skin as an array of small, regularly spaced beams, with a depth of treatment ranging from 1.5 to 3 mm into the dermis. This fractional heating creates a lattice of hyperthermic islets, with each islet surrounded by unaffected tissue. Other devices that employ fractional technology are the Palomar® 1540 Fractional Handpiece, the Reliant Fraxel® SR Laser and similar devices by ActiveFX, Alma Lasers, Iridex, and Reliant Technologies. These devices are sold to and used by professionals, such as doctors.

However, there is no effective fractional device that can be used by a consumer in a non-medical and/or non-professional setting. Fractional systems designed for use by professionals are large, expensive, complex, generally utilize expensive cooling systems, and are not generally safe for use by non-professionals. Some systems, such as certain Reliant Fraxel systems, require the application of anesthetics and/or dyes.

On the other hand, most light-based treatment devices that are currently available to consumers are not adequate to provide efficacious photocosmetic treatments. Such devices are typically too simplistic and have very low power. Such devices are either not efficacious or have very limited and unsatisfactory efficacy. Thus, there is a need for a fractional photocosmetic device that can be utilized by a consumer in a non-professional setting, such as the home. Such a device would preferably perform one or more photocosmetic treatments; would be efficacious; would be durable; would be relatively inexpensive; would have a simpler design relative to current fractional devices; would be smaller than existing professional devices; would be safe for use by non-professionals; and/or would not be painful to use. EMR

### SUMMARY OF THE INVENTION

The inventors have resolved the various technical challenges associated with the creation of an effective fractional photocosmetic device for use by a consumer in a non-medical and or non-professional setting. Thus, embodiments of such devices are disclosed herein that have one or more of the following attributes: capable of performing one or more cosmetic and/or dermatological treatments; efficacious for such treatments; durable; relatively inexpensive; relatively simple in design; smaller than existing professional devices (with some embodiments being completely self-contained and hand-held); safe for use by non-professionals; and/or not painful to use (or only mildly painful). While each of these attributes is desirable, embodiments of the invention need not have all such attributes, but may instead have one or a subset of these attributes.

The inventors have further discovered that the frequent periodic application of relatively lower intensity treatments than existing professional treatments, e.g., treatments having larger pitch between islets, fewer islets per unit area and/or volume of tissue, and/or relatively lower power density applied per treatment islet, provides improved efficacy over time. Thus, in some aspects of the invention, methods for using fractional devices are disclosed.

In one aspect, the invention discloses a handheld photocosmetic device for performing fractional treatment of tissue by a user including a housing, an EMR source disposed in the housing, and an EMR delivery path within the housing and optically coupled to the light source. The EMR delivery path is configured to apply EMR generated by the EMR source to a plurality of discrete locations located within a treatment area of the tissue and wherein a total area of the plurality of discrete locations is less than the treatment area. The device is configured to be self-contained within or about the housing such that substantially the entire device can be handheld by the user during operation. The EMR delivery path can include a plurality of microlenses. The discrete locations can be distributed according to a predetermined or random pattern. The total area of the plurality of locations is between approximately 1 and 90 percent of the treatment area, between approximately 30 to 90 percent of the treatment area, or between approximately 50 to 80 percent of the treatment area. In some embodiments, a lotion dispenser can be coupled to the housing.

In some embodiments, a power source can be coupled to the housing and can be in electrical communication with the EMR source, wherein the power source is configured to supply power to the EMR source. The device can include an electrical cord in electrical communication with the EMR source and configured to supply power to the EMR source. In preferred embodiments, the power source includes a battery. The batter can be rechargeable.

In some embodiments, the EMR delivery path comprises an optical scanner. The scanner can include at least one optical fiber having an input port adapted to receive EMR from the EMR source and having an output port through which EMR can be delivered to the locations. The scanner can further include a scanning mechanism coupled to the output port of the fiber for moving the output port to direct EMR to the locations. The scanning mechanism can be optically coupled to the output port of the fiber, and further comprises one or more rotatable mirrors for directing the EMR to the locations. In some embodiments, the scanning mechanism has at least one piezoelectric scanner element. For example, the piezoelectric scanner element can be an adjustable multilayer piezoelectric device. The scanner comprise also include at least one stepper motor.

In other embodiments, the device further includes optics coupled to the output port for shaping the EMR passed through the output port.

In another aspect, the handheld photocosmetic device can further include controller for controlling the EMR source in substantial synchrony with the movement of the fiber's output port to effect delivery of EMR to the locations. The controller can selectively activate the EMR source. In some embodiments, the controller selectively blocks EMR emitted from the source from entry into the fiber.

In yet other embodiments, the handheld photocosmetic device can further include an optical coupler disposed between the EMR source and the optical fiber for directing light from the source into the fiber. The coupler can have one or more focusing optical elements for focusing EMR from the source into the fiber. The focusing elements focus the EMR into the fiber at a numerical aperture in a range of about 0.5 to about 3. The coupler can include a connector for selectively connecting a selected EMR source and a selected optical fiber. The EMR source and the input port of the optical fiber are aligned such that at least about 60% of EMR energy, or at least about 70% of EMR energy, or preferably at least 80% of EMR energy, generated by the source is coupled into the optical fiber.

In another aspect, the invention discloses a safety system for the handheld photocosmetic device having one or more sensors for sensing one or more operating parameters of the device. At least one of the sensors can include a contact sensor for sensing contact between an EMR-emitting end of the device and the skin. The safety mechanism can, for example, inhibit delivery of light to the skin if the contact sensor senses contact below a minimum contact threshold. The minimum contact threshold is a contact area greater than about 60%, or about 70%, or about 80% of an area of the EMR-emitting end. The contact sensor can be selected from the group comprising conductance sensors, piezoelectric sensors, and mechanical sensors. In some embodiments, the safety system inhibits delivery of EMR energy exceeding a predefined threshold to a skin location with which an EMR-emitting end of the device is in contact. The safety system can inhibit delivery of EMR exceeding a predefined threshold to the skin during a treatment session, wherein a treatment session comprises a temporal period following activation of the device.

In some embodiments, the safety system includes a controller tracking an amount of EMR energy being applied to a skin location, the controller inhibiting delivery of EMR to the skin upon the energy reaching the threshold. The controller can be configured to de-activate the source to inhibit delivery of EMR to the skin.

The EMR source of the handheld photocosmetic device can generate EMR with one or more wavelengths in a range of about 300 nm to about 11,000 nm, and preferably in a range of about 300 nm to about 1800 nm. The EMR source can be a coherent light source, such as a single diode laser, a plurality of diode lasers, or at least one diode laser bar. In other embodiments, the light source is an incoherent light source. For example, the incoherent light source can be selected from the group consisting of light emitting diodes (LED), arc lamps, flash lamps, fluorescent lamps, halogen lamps, and halide lamps.

In another aspect, the invention discloses a handheld photocosmetic device including a housing with at least two separable modules one of which contains the EMR source and the other contains the EMR delivery mechanism. The modules include mating connectors for removably and replaceably engaging to one another. In some embodiments, the device includes a sensor system capable of sensing the type of EMR source and indicating the type to the scanner. The device can also include a cooling mechanism thermally coupled to the EMR source. The cooling mechanism can include a thermoelectric cooler for extracting heat from the EMR source, and/or a thermal mass for extracting heat from the EMR source.

In some embodiments, the handheld photocosmetic device includes a rechargeable power supply disposed in the housing. A docking station is disclosed that is adapted for coupling to the housing and comprises circuitry for recharging the power supply.

In another aspect, the invention discloses a photocosmetic system, including a handheld portion extending from a proximal end to a distal end, an EMR source disposed in the handheld portion, a plurality of EMR-delivery modules, wherein each of the modules is adapted for removable and replaceable coupling to the distal end of the handheld portion for delivery of light from the source to a plurality of distributed discrete skin locations. Each of the light-delivery module provides a different pattern of the discrete locations. The handheld portion and the modules can include mating connectors for removably and replaceably engaging to one another, such that a combination of the handheld portion and each module provides a handheld device. The patterns formed by the modules vary in area, pitch, shape, and/or focal depth. The proximal end is capable of being coupled to a docking station. The docking station comprises circuitry for recharging the power source. The handheld portion can include a power source.

In yet another aspect, the invention discloses a photocosmetic device including a housing extending from a proximal end to a distal end, a plurality of light sources disposed in the housing configured to direct light through the distal end of the housing to a plurality of separated discrete skin locations, a motion sensor mounted to the housing to sense a speed of movement of the distal portion to the skin, and a controller in communication with the motion sensor and the light sources. The controller can control the sources based on the speed so as to direct light from the source to a plurality of separated discrete skin locations. In some embodiments, the controller can control the selective activation of the sources. In other embodiments, the sources are pulsed and the controller controls the repetition rate of the pulses.

The invention also discloses a method of maintaining improved skin appearance comprising regular application of the EMR from the device between 1 and 3 times a day, with 0 to 7 days intervals between treatment days.

In another aspect, a method for performing fractional treatments of tissue using a handheld photocosmetic device is disclosed comprising irradiating in a first treatment a plurality of separated treatment spots within a target area of tissue with EMR, wherein the total area of the plurality of treatment spots is less than the area of the target area; irradiating in a second treatment a second plurality of separated treatment spots within the target area of tissue with EMR, wherein the total area of the second plurality of treatment spots is less than the area of the target area. The second irradiating step occurs after the first irradiating step and wherein at least the second irradiating step is performed using a self-contained handheld photocosmetic device. The irradiation steps can be repeated between one to five times per day, preferably one to three times per day. An interval of no treatment of between zero and seven days can exist between treatment days. The irradiation steps include delivering EMR radiation in a range of about 2 mJ to 30 mJ per treatment spot, preferably in a range of about 3 mJ to 20 mJ per treatment spot, or in a range of about 4 mJ to 10 mJ per treatment spot. The plurality of treatment spots can be treated with EMR between about 2 to 10 times per treatment. The method can include irradiating a density of treatment spots ranging from about 100/cm² to about 700/cm² during an irradiation treatment. The intensity of irradiation can be adjusted between irradiation steps. In some embodiments, the intensity of irradiation is adjusted by a profession. In other embodiments, the intensity is adjusted by the user. Professional EMR treatments can be used in conjunction with the disclosed method. The method can be used to maintain and improve the benefits obtained through professional EMR treatments.

### BRIEF DESCRIPTION OF THE DRAWINGS

The following drawings are illustrative of embodiments of the invention and are not meant to limit the scope of the invention as encompassed by the claims.

FIG. 1 is a schematic depiction of an exemplary handheld photocosmetic device according to one embodiment of the invention;

FIG. 2A is a schematic view a two-dimensional rectangular lattice of discrete location or islets that can be created at a skin surface or at a selected depth from the skin surface;

FIG. 2B is a schematic view a two-dimensional spiral lattice of discrete location or islets that can be created at a skin surface or at a selected depth from the skin surface;

FIG. 3A is a schematic depiction of an exemplary handheld device handheld photocosmetic device;

FIG. 3B is a more detailed depiction of the device of FIG. 3A;

FIG. 3C is an exploded view of the device of FIG. 3B;

FIG. 3D is an enlarged view of the fiber translation mechanism showing also the guide for the fiber of the device of FIG. 3A;

FIG. 3E is an enlarged front view of a spiral scanning mechanism and captive contact sensors used in the device of FIG. 3A;

FIG. 3F is a schematic view of a microoptic that can be formed or attached to the distal end of the optical fiber of the device of FIG. 3A to provide shaping and/or focusing of the output beam.

FIG. 4A is a schematic view of an EMR source used in the device of FIG. 3A in which the EMR emitter is mounted on a mount;

FIG. 4B is a schematic view of the EMR source of FIG. 4A coupled to an optical fiber;

FIG. 4C is a perspective view of the EMR source of FIG. 4A mounted on a mount coupled to a cooling system;

FIG. 5A schematically depicts an alternative embodiment of a thermal management system for controlling the temperature of an EMR source;

FIG. 5B schematically depicts another embodiment of a thermal management system for controlling the temperature of an EMR source;

FIG. 6 is a schematic depiction of electronics of the device of FIG. 3A;

FIG. 7A is a side cross-section view showing one method of optical coupling of the EMR from the device to an optical fiber of the device of FIG. 3A coupled to the EMR source using a V-groove;

FIG. 7B is a side view of another mechanism to optically couple an EMR source to an optical fiber that may be used in other embodiments;

FIG. 7C is a perspective view of another mechanism to optically couple an EMR source to an optical fiber that may be used in other embodiments;

FIG. 7D is a side view of another mechanism using a fiber bundle to optically couple an EMR source to an optical fiber that may be used in other embodiments;

FIG. 7E is a bottom view of the embodiment of FIG. 7D;

FIG. 7F is an enlarged, side view of a distal end of another embodiment of a device employing a fiber bundle;

FIG. 8 is a side perspective view of an X-Y linear translation system for use in other embodiments;

FIG. 9 is a schematic depiction of an alternative embodiment of a handheld photocosmetic device with an EMR delivery mechanism comprising two rotatable mirrors;

FIG. 10 a schematic depiction of an alternative embodiment of a handheld photocosmetic device with a plurality of microlenses;

FIG. 11A is a schematic depiction of a an alternative embodiment employing a modular handheld device;

FIG. 11B is a schematic depiction of module of the modular handheld device of FIG. 1A;

FIG. 12A is an exploded view of an alternative embodiment of a modular handheld device;

FIG. 12B is a side perspective view of the assembled modular handheld device of FIG. 12A;

FIG. 12C is an enlarged cross-sectional view of the module of the modular handheld device of FIG. 12A;

FIG. 13A is a schematic view of another embodiment of a modular handheld device;

FIG. 13B is a schematic view of two the separated modules of FIG. 13A;

FIG. 14A is a side perspective view of another embodiment of a handheld dermatological device that includes a plurality of EMR sources;

FIG. 14B is a front perspective view of the device of FIG. 14A EMR;

FIG. 14C is a perspective view of a diode laser bar used in the device of FIGS. 14A and 14B;

FIG. 15A is a depiction of a mechanical sensor suitable for use with the device of FIG. 14A;

FIG. 15B is a depiction of an alternative optical sensor suitable for use with alternative embodiments;

FIG. 16A is a depiction of an exemplary pattern in which EMR is applied to form a plurality of continuous linear segments;

FIG. 16B is a depiction of an exemplary alternative pattern in which EMR is applied to form a plurality of linear segments formed by sets of discrete islets; and

FIG. 17 is a schematic depiction of another embodiment of a handheld photocosmetic device, which includes a lotion dispenser.

### DETAILED DESCRIPTION

When using electromagnetic radiation (EMR) to treat tissues, there are substantial advantages to producing lattices of EMR-treated discrete locations or islets in the tissue rather than large, continuous regions of EMR-treated tissue. The lattices are periodic or aperiodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.” By producing EMR-treated islets rather than continuous and/or uniform regions of EMR-treatment, more EMR energy can be delivered to an islet without producing a thermal islet or damage islet, and/or the risk of bulk tissue damage can be lowered.

EMR-treated islets can also be formed within an area or volume of treated tissue, for example, where the entire tissue area and/or volume is treated with a relatively lower intensity of EMR having a same or different wavelength while the islets are formed by treating portions of the area and/or volume using EMR having a higher intensity. One skilled in the art will recognize that many combinations of parameters are possible that will result in such local maxima of EMR-treatment within the tissue.

When using electromagnetic radiation (EMR) to treat tissues, whether for purposes of photodynamic therapy, photobiomodulation, photobiostimulation, photobiosuspension, thermal stimulation, thermal coagulation, thermal ablation or other applications, there are substantial advantages to producing lattices of EMR-treated islets in the tissue rather than large, continuous regions of EMR-treated tissue. The EMR-treated tissues can be any hard or soft tissues for which such treatment is useful and appropriate, including but not limited to dermal tissues, mucosal tissues (e.g., oral mucosa, gastrointestinal mucosa), ophthalmic tissues (e.g., retinal tissues), neuronal tissue, vaginal tissue, glandular tissues (e.g., prostate tissue), internal organs, bones, teeth, muscle tissue, blood vessels, tendons and ligaments.

The lattices are periodic or aperiodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.”

By producing EMR-treated islets rather than continuous regions of EMR-treatment, untreated regions (or differently- or less-treated regions) surrounding the islets can act as thermal energy sinks, reducing the elevation of temperature within the EMR-treated islets and/or allowing more EMR energy to be delivered to an islet without producing a thermal islet or damage islet and/or lowering the risk of bulk tissue damage. Moreover, with respect to damage islets, it should be noted that the regenerative and repair responses of the body occur at wound margins (i.e., the boundary surfaces between damaged and intact areas) and, therefore, healing of damaged tissues is more effective with smaller damage islets, for which the ratio of the wound margin to volume is greater.

The percentage of tissue volume, which is EMR-treated versus untreated (or differently- or less-treated) can determine whether optical islets become thermal islets, damage islets or photochemical islets. This percentage is referred to as the “fill factor”, and can be decreased by increasing the center-to-center distance(s) of islets of fixed volume(s), and/or decreasing the volume(s) of islets of fixed center-to-center distance(s). For a given treatment, the total area of the discrete treatment spots or islets within the treated area is less than the treatment area itself. Similarly, the total volume of the discrete treatment islets within the volume to be treated is less than the volume to be treated itself.

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Additionally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The embodiments described below provide improved devices and systems for producing lattices of EMR-treated islets in tissues, and improved cosmetic applications of such devices and systems.

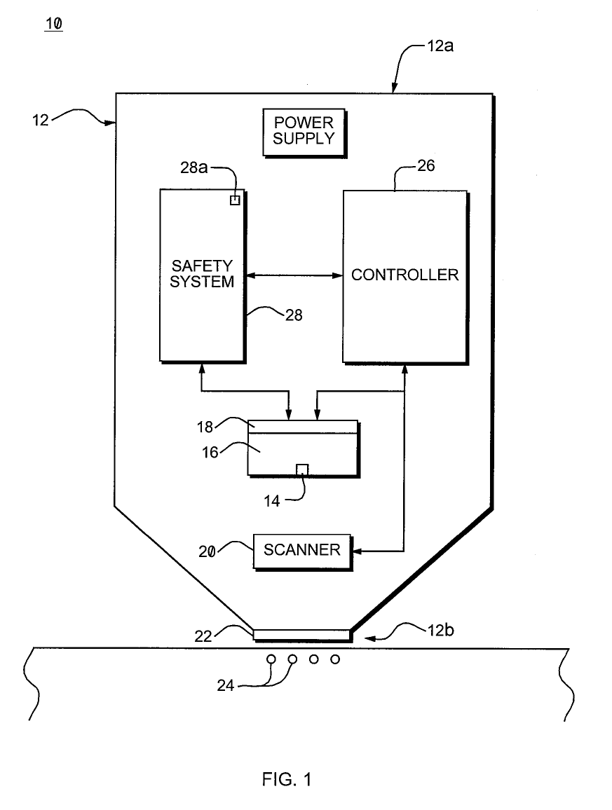

FIG. 1 schematically depicts an exemplary photocosmetic device 10 according to one embodiment of the invention that includes a handheld housing 12 in which various components of the device, such as optical and electrical components, are disposed. The housing 12 extends from a proximal end 12a to a distal end 12b, through which electromagnetic radiation (“EMR”) can be applied to the skin. The exemplary device 10 includes an EMR source 14 that generates EMR with one or more wavelengths in a desired range. In some implementations, the EMR source 14 can be a diode laser, though a variety of other EMR sources, such as those listed further below, can be also employed. The EMR source can be thermally coupled to a heat sink 16, which is in turn thermally coupled to a cooler 18 that extracts heat from the source via the heat sink to maintain the operating temperature of the source within an acceptable range. As discussed in more detail, a variety of coolers, such as a thermoelectric cooler or a thermal mass, can be employed.

An EMR delivery mechanism 20 disposed in the housing and in optical communication with the EMR source 14 receives the EMR generated by the source and delivers the EMR, through an EMR transmissive window 22 (e.g., a sapphire window), to a plurality of distributed discrete skin locations 24. In this implementation, the EMR delivery mechanism is an optical scanner that scans an EMR beam generated by the source 14 over the skin so as to deliver optical energy to the discrete skin locations 24, as discussed further below. In other implementations, rather than utilizing a scanner, other mechanisms, e.g., a plurality of microlenses, can be employed to direct the EMR to a plurality of distributed discrete skin locations.

The device 10 further includes a controller 26 that controls activation and deactivation of the source, and can provide other functionality, such as controlling the EMR delivery system 20 (e.g., actuating the delivery system and controlling the scanning rate of EMR over the skin), as discussed further below.

In use, the distal portion 12b of the device 10 can be placed in contact with, or in proximity to, the surface of a skin portion and the device can be activated to apply EMR to the discrete locations, such as islets 24. In some implementations, the controller 26 can selectively activate the EMR source 14 (e.g., periodically activate the source to cause to source to emit a plurality of temporally separated pulses) in coordination with the scanner so as to effect delivery of the EMR to the plurality of separate discrete locations 24. In some implementations, once activated, the EMR source can provide a train of laser pulses. In such implementations, the controller can adjust the scanning speed of the EMR over the skin based on the repetition rate of the pulses (based on the time interval between consecutive pulses) to effect delivery of the EMR pulses to the discrete skin locations. In other implementations, a shutter can modulate the intensity of the EMR emitted by the continuous-wave (CW) or a quasi-continuous (QCW) source (e.g., it can periodically block an EMR beam emitted by the source) in coordination with the scanner to effect delivery of the EMR to the discrete locations.

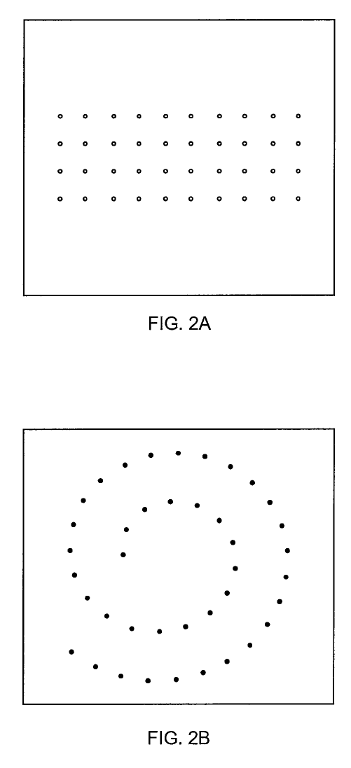

The plurality of the discrete locations to which the EMR is applied can correspond to any desired pattern. By way of example, as shown in FIG. 2A, the discrete locations 24 can lie at a selected depth from the skin surface (e.g., the depths from the surface of a tissue can vary from 0-4 mm, 0-50 μm, 50-500 μm, or 500 μm-4 mm, as well as sub-ranges within these ranges) as a two-dimensional rectangular lattice (e.g., a lattice of 10×4 skin locations in this case), or a square lattice in other cases. Alternatively, as shown in FIG. 2B, the discrete locations can be distributed in accordance with a spiral pattern. In other cases, the plurality of the discrete locations can be distributed within a three-dimensional skin portion, e.g., through a plurality of skin layers each of which is located at a different skin depth. In many embodiments, the skin locations to which the EMR delivered are separated from one another by skin portions that are not exposed to EMR from the source, or other differently irradiated.

Referring again to FIG. 1A, the device 10 can also include a safety system 28 that can ensure that one or more operating parameters of the device remain within acceptable ranges, and that the device is utilized in a safe manner. By way of example, the safety system 28 can include a contact sensor (not shown) that senses the degree of contact between the output window 22 and the skin. In some implementations, if the sensed contact is below a predefined threshold, the safety system inhibits activation of the EMR source, e.g., by sending a signal to the controller 26 that would in turn inhibit the activation of the light source, or would deactivate the source if it is emitting EMR. By way of example, if no contact is detected or if the fraction of the area of the window 22 that is in contact with the skin is less than a predefined threshold, e.g., less than about 20%, 30%, 50%, 70%, or 80%, the source is not activated. In some applications, it may be preferable for the predefined contact threshold to be about 70%. In some cases, the contact sensor can detect not only direct physical contact between the output window 22 and the skin but can also sense whether the output window is sufficiently close to the skin—though not touching the skin—to allow safe operation of the device. For example, if more than a predefined portion of the window (e.g., more than 80%) is separated from the skin by less than a predefined threshold (e.g., 1-10 microns), the source can be activated. Otherwise, the activation of the source is inhibited. A variety of contact sensors can be employed. By way of example, the sensor can be mechanical, optical, magnetic, electronic, conductive, and/or piezoelectronic.

In some embodiments, the device can also include a speed sensor. For example, the sensor can determine the speed of movement of the device across the target area of the patient's skin. The device can include circuitry in communication with the sensor for controlling the source based on the speed of movement across the target area of the patient's skin, such that islets of treatment are formed on the target area of the patient's skin. For example, the circuitry can communicate the speed of the device to the controller 26 that can selectively activate the EMR source 14 in coordination with the scanner so as to effect delivery of the EMR to the plurality of separate discrete locations 24 based on the speed. In some aspects, the sensor can be a capacitive imaging array or an optical encoder. In some embodiments, a kinematic motion sensor can used alone or to supplement an optical motion sensor. The kinematic motion sensor can, for example, be a wheel which turns the output window 22 is moved over the skin surface to provide a signal to the controller 26 indicative of scan velocity. In some embodiments, the source and/or the scanner may be controllable based on speed of movement across the skin as measured by a motion sensor, or a temperature measured at the skin by a temperature sensor or a temperature of the source measured by a temperature sensor.

A number of types of speed sensors can be used to measure the device speed relative to the skin surface. For example, the speed sensor can be an optical mouse, a laser mouse, a wheel/optical encoder, or a capacitive imaging array combined with a flow algorithm similar to the one used in an optical mouse. A capacitive imaging array can be used to measure both device speed and to create an image of the treated area. Capacitive imaging arrays are typically used for thumbprint authentication for security purposes as well as various other electronic products such as laptop computers. However, a capacitive imaging array can also be used to measure the device speed across the skin surface. By acquiring capacitive images of the skin surface at a relatively high frame rate (for example, 100-2000 frames per second), a flow algorithm can be used to track the motion of certain features within the image and calculate speed.

Such sensors and applications useful in understanding and practicing the embodiments described herein are disclosed more fully in U.S. Pat. No. 6,273,884 entitled Method and Apparatus for Dermatology Treatment, Issued Aug. 14, 2001, which is incorporated herein by reference. Additional disclosure related to motion sensors and temperature sensors are described in greater detail in U.S. Pat. No. 7,204,832 entitled “Cooling system for a photo cosmetic device”, U.S. Pat. No. 7,135,033 entitled “Phototreatment device for use with coolants and topical substances”, U.S. Pat. No. 6,508,813 entitled “System for electromagnetic radiation dermatology and head for use therewith.” and co-pending U.S. application Ser. Nos. 11/097,841, 11/098,036, 11/098,015, 11/098,000, entitled “Methods and products for producing lattices of EMR-treated islets in tissues, and uses therefore” filed Apr. 1, 2005, which are hereby incorporated by reference.

Many other sensors and feedback mechanisms are possible. For example, the device can be preprogrammed with treatment profiles for one or more specific user. To identify the individual user, a code or biometric identifier (e.g., fingerprint) can be used.

Many different diagnostic sensors can also be used. For example, sensors to measure skin elasticity, pigmentation, surface roughness, or other characteristics of tissue can be used. These sensors can provide feedback within the device or to the user to indicate the status of, or the control of, the treatment. One exemplary sensor could be a CCD camera installed in proximity to the aperture to provide an image for analysis to determine if the area of tissue to be treated is appropriate for treatment. For example, if a device is designed to treat pigmented or vascular lesions, and the device determines from the image that the area of skin lack sufficient indicia of such a lesion, the device could be programmed to not fire until a suitable area is contacted. Similarly, a feedback signal, e.g., a vibration and/or tone, could be issued to the user to indicate that the tissue in the proximity of the device is not suitable for treatments.

The device could include one or more timing mechanisms to assist with treatment. For example, a device could include a timer that prevents the device from being used within a specified time following a treatment. The device could include a feedback mechanism to remind a user that a subsequent treatment is required/appropriate. For example, the device could be set or programmed to issue a series of tones for a particular duration of time (such as one hour) beginning at a certain time of day (e.g., 6:00 a.m.) Thus, the user could program a treatment reminder that coincides with time that the user would typically perform the treatment and is typically.

Additional sensors and feedback mechanisms can be employed to improve safety of the device. As discussed in more detail below, the safety system 28 can also include other sensors for monitoring one or more parameters of the device. For example, a temperature sensor 28a can monitor the ambient temperature within the device and/or monitor the temperature of the EMR source. If the temperature detected by the sensor exceeds a predefined value, the safety system can send a signal to the controller to cause the controller to deactivate the EMR source. By way of example, a temperature sensor can be mounted to or embedded in the distal portion 12b of the device 10 to assure that the device 10 is not used when its surface temperature is outside a selected range. The sensor can be a thermocouple embedded in the outer surface of the device 10, or within the device 10 which, for example, couples to an LED or other suitable display mounted on the device; or may be an adhesive strip the color of which changes with temperature in the relevant range, the color of the strip being indicative of surface, ambient temperature within the device and/or the temperature of the EMR source. For example, the temperature of the system thermal capacitance can be monitored with a thermistor that can be integrated onto the circuit board, as discussed further below. In addition, other suitable sensors could also be utilized. The temperature sensor can also send a signal to the lotion dispenser (discussed below) causing a valve to release lotion, send a signal to control the activation of the thermoelectric cooler (TEC), and/or send a signal to the LED indicators indicating, for example, overheating of the device, as discussed further below. Examples of temperature sensors can be found in U.S. Pat. No. 6,508,813 entitled “System for electromagnetic radiation dermatology and head for use therewith,” U.S. Pat. No. 6,648,904 entitled “Method and apparatus for controlling the temperature of a surface,” and U.S. Pat. No. 6,878,144 entitled “System for electromagnetic radiation dermatology and head for use therewith,” which are hereby incorporated by reference.

A variety of other safety mechanisms can also be included in hardware and/or software, as discussed further below. For example, one such safety mechanism can monitor the EMR energy deposited during a session (defined, e.g., as a predefined time interval following the initial activation of the EMR source after the device is switched on) and deactivate the source if the total energy delivered to the skin would begin to exceed a pre-defined threshold.

Referring again to FIG. 1A, the device 10 further includes a rechargeable power supply 30 (e.g., a rechargeable battery) that can provide power to various components of the device. The handheld device 10 can be engaged with a docking station that allows charging the rechargeable power supply, e.g., in a manner discussed further below. Alternatively, a power chord to be plugged into an electrical outlet can be used to supply power to the device. This may be preferable in embodiments that require sustained power over a longer period, higher peak power, and/or higher average power, and additionally may help to save space in embodiments in which may require larger amounts of cooling, and therefore, a larger cooling system.

The EMR applied to the skin can include a variety of electromagnetic wavelengths, e.g., wavelengths ranging from about 0.29 microns to about 12 microns. Although smaller wavelengths are also possible, wavelengths greater than 0.29 are preferably used due to the potential risks associated with radiating tissue with ultraviolet light. A preferred range of wavelengths for many embodiments described herein is about 1.1 microns to about 1.85 microns, with wavelengths ranging from about 1.54 microns to about 1.06 microns being preferred. In some implementations, the EMR source provides EMR with wavelengths that are less likely to cause retinal damage, e.g., wavelengths that are absorbed by water (e.g., wavelengths in a range of about 600-680 nm, or have a wavelength that is predominately red, or the spectrum of the light is in the range of or around the absorption peaks for water, for example, 970 nm, 1200 nm, 1470 nm, 1900 nm, 2940 nm).

The EMR source can be a variety of coherent and non-coherent EMR sources, which can be employed individually or in combination with other sources. In some embodiments, the EMR source is a laser, such as a solid-state laser, a dye laser, a diode laser, or other coherent light sources. For example, the EMR source can be a diode laser, a neodymium (Nd) laser, such as a Nd:YAG laser, a chromium (Cr) or a Ytterbium (Yt) laser. Another example of a coherent EMR source is a tunable laser. For example, a dye laser with non-coherent or coherent pumping that provides wavelength-tunable emission can be employed. Typical tunable wavelength bands cover a wavelength range of about 400 to about 1200 nm with a bandwidth in a range of about 0.1 to about 10 nm. Further, mixtures of different dyes can provide multi-wavelength emission. In some embodiments, the EMR source is a fiber laser. The wavelength range of such a laser is typically in a range of about 1100 nm to about 3000 nm. This range can be extended with the help of second harmonic generation (SHG) or an optical parametric oscillator (OPO) optically connected to the fiber laser output. In other embodiments, diode laser can be used to generate EMR with wavelengths, e.g., in a range of about 400-100,000 nm. In some embodiments in which a system of the invention is employed for non-ablative skin remodeling, the EMR from the source can be applied to the skin while cooling the surface to prevent damage to the epidermis.

Alternatively, in some embodiments, non-coherent EMR sources, such as incandescent lamps, halogen lamps, light bulbs a linear flash lamp, or an arc lamp can be used. By way of example, monochromatic lamps, such as hollow cathode lamps (HCL), electrodeless discharge lamps (EDL), which generate emission lines from chemical elements, can be utilized.

Further, although the EMR is typically applied in a pulsed manner, it can also be applied in other ways, including continuous wave (CW) and quasi-continuous wave (“QCW).

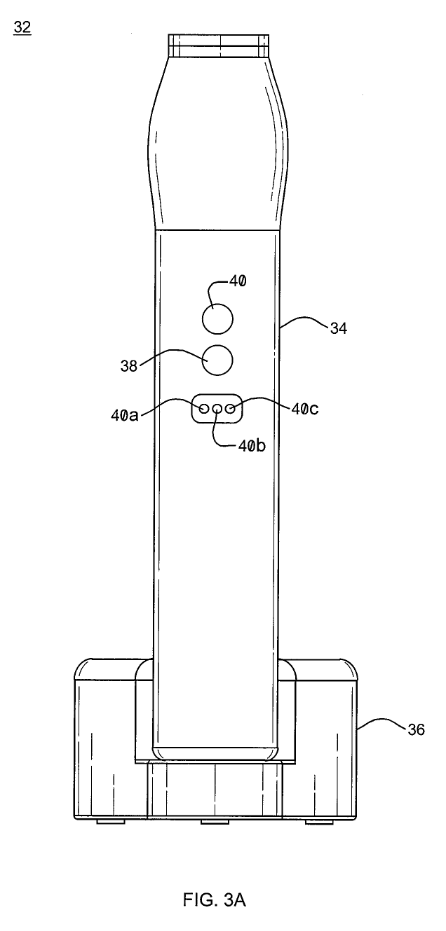

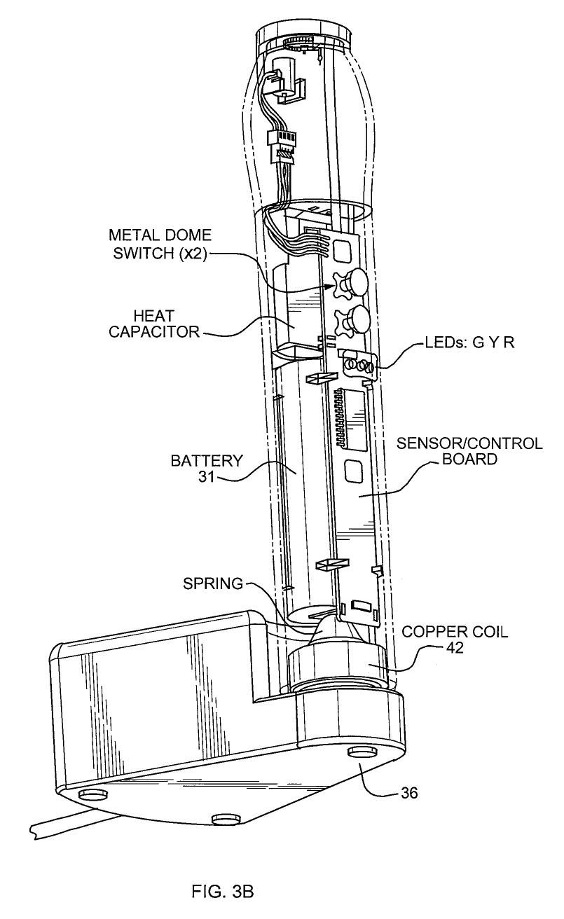

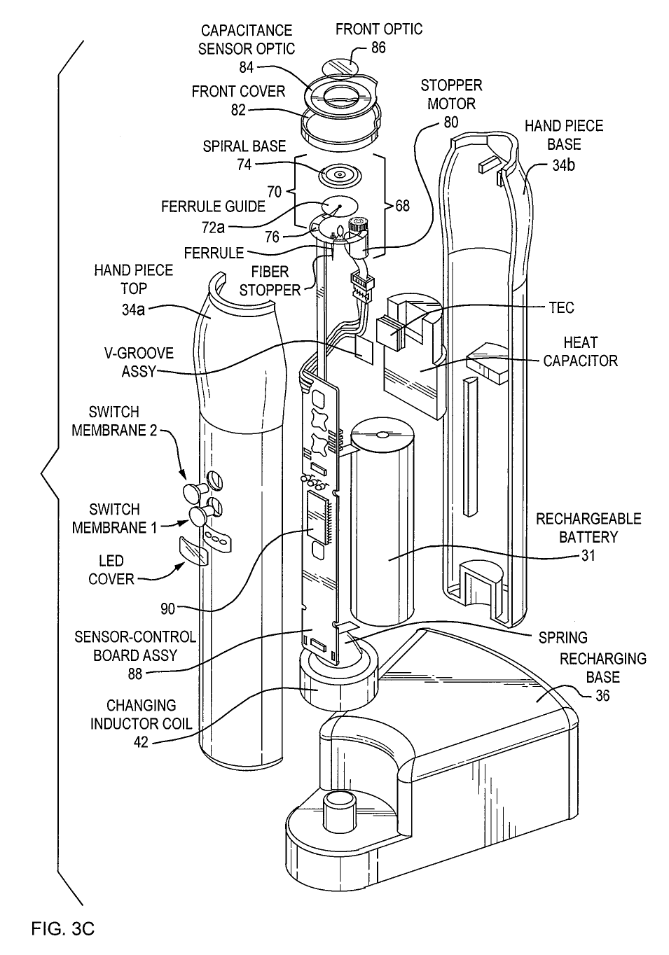

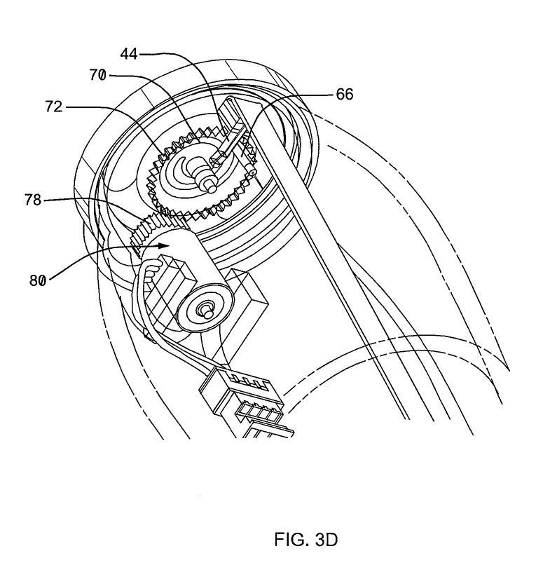

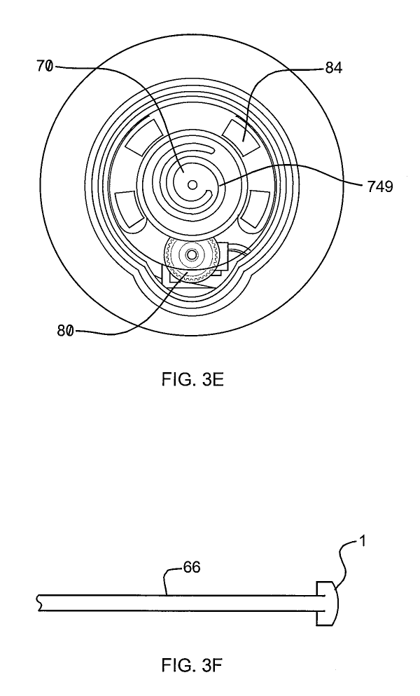

A handheld dermatological device of the invention can be implemented in a variety of different ways. By way of further illustration, FIG. 3A, 3B, 3C, 3D and 3E schematically depict a handheld photocosmetic device 32 according to one embodiment of the invention that includes a handheld housing 34, 34A, 34B that can be engaged with a docking station 36, e.g., to charge a rechargeable battery of the device. In use, the handheld device can be removed from the docking station and utilized to apply EMR to the skin in a manner discussed above and further elaborated below. A button 38 disposed on the housing so as to be accessible to a user allows switching on the device, and another button 40 allows activating the device's EMR source to apply EMR to the skin. A plurality of LED indicators 40a, 40b, 40c provide the user with information about characteristics of the device, such as that a fault has occurred (e.g., overheating, low battery voltage), that the system is ready for use, that the system is on, that the battery is charging, or that battery charging is complete.

The exemplary device 32 further includes a rechargeable battery 31 for supplying power to its various components, which can be charged through inductive coupling, via a copper coil 42, with charging circuitry disposed in the docking station 36. The device 32 further includes an EMR source 44, a diode laser in this example, which provides EMR with one or more wavelengths in a desired range.

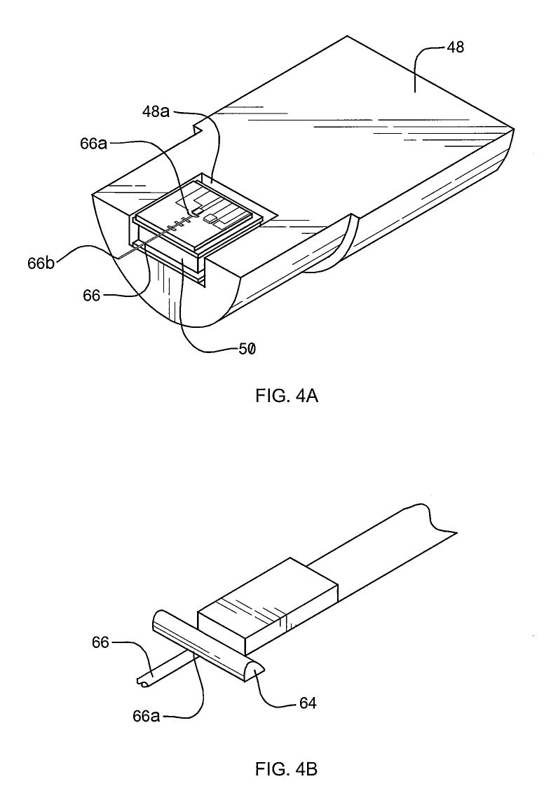

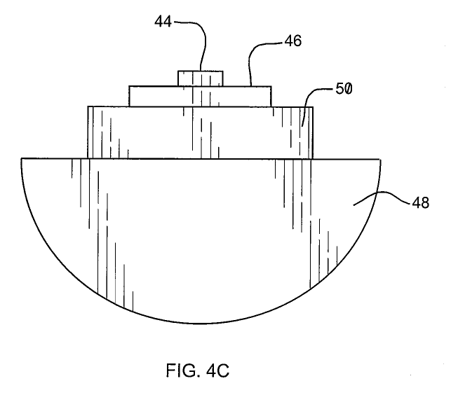

With reference to FIGS. 4A, 4B and 4C, the diode laser 44 is mounted on a mount 46, in this case a submount or platform of the larger assembly. The mount is preferably formed of a thermally conductive material. The mount 46 can in turn be disposed in a recess 48a of a heat sink 48, e.g., a heat capacitor in this exemplary implementation. A thermoelectric cooler (“TEC”) 50, which is in thermal coupling with the heat capacitor 48 as well as the mount 46, can remove heat generated by the EMR source to ensure that its temperature remains within an acceptable range (e.g., below about 60C.).

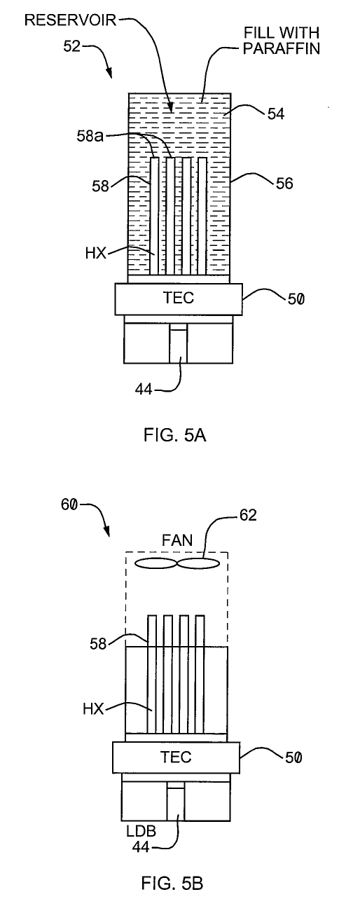

In some implementations, the thermal management of the EMR source is achieved by utilizing a TEC in conjunction with flow of a cooling fluid (e.g., air flow) and/or a thermal mass. For example, FIG. 5A schematically depicts a thermal management system 52 for controlling the temperature of the EMR source 44, which includes the TEC 50 in thermal contact with the EMR source. The TEC is in thermal communication with a thermal mass 54 (e.g., paraffin or water) contained in a reservoir 56. The thermal mass helps in dissipating the heat extracted by the TEC from the source. A thermally conductive element 58 disposed in the reservoir provides a thermal link between the TEC and the thermal mass within the reservoir. The thermally conductive element 58 includes a plurality of fins 58a that increase the area of contact between the element and the thermal mass within the reservoir, thereby facilitating the transfer of heat between TEC and the thermal mass. FIG. 5B schematically depicts another thermal management system 60 for controlling the temperature of the EMR source 44 in which the TEC 50 removes heat from the source. In this case, the thermally conductive element 58 facilitates transfer of heat away from the TEC to be more readily dissipated by an air flow generated by a fan 62.

In other cases, a phase change material can be utilized to remove heat from the source via phase change. Examples of such phase change materials and systems for their use in cooling an EMR source can be found, for example, in U.S. Pat. No. 7,135,033 entitled “Phototreatment Device for Use with Coolants and Topical Substances” which is incorporated by reference.

Referring again to FIGS. 3C, 3D, 4A, and 4B, the EMR emitted by the source is coupled, via an optical coupler 64, discussed in more detail below, to an optical fiber 66 via a proximal end 66a thereof. A distal end 66b of the fiber is engaged with a scanning mechanism 68 that can physically move the fiber's distal end over the skin, e.g., along a spiral path in this implementation.

With reference to FIGS. 3C, 3D and 3E, in this exemplary embodiment, the scanning mechanism 68 includes a fiber guide 70 to which the distal tip of the optical fiber 66 can be coupled so as to be moved along a spiral path. More particularly, the fiber guide 70 includes a gear 72 having an opening 72a for receiving the fiber's distal end and a guiding element 74, which is disposed within a recess in the gear 72. The guiding element 74 includes a spiral groove 74a along which the distal end of the fiber can be moved. More particularly, a ferrule 76 can engage the gear 72 with a gear 78, which can be rotated by a stepper motor 80. The rotation of the gears can cause the movement of the fiber tip through the spiral groove.

With continued reference to FIG. 3C, an annular front cover 82, which is adapted to receive a contact sensor 84 (e.g., a capacitance contact sensor) having an annular shape, surrounds the scanning mechanism. The annular sensor provides a seat for an EMR transmissive output window 86 (also referred to herein as the front optic) through which the EMR emanating from the fiber tip can be applied to the skin.

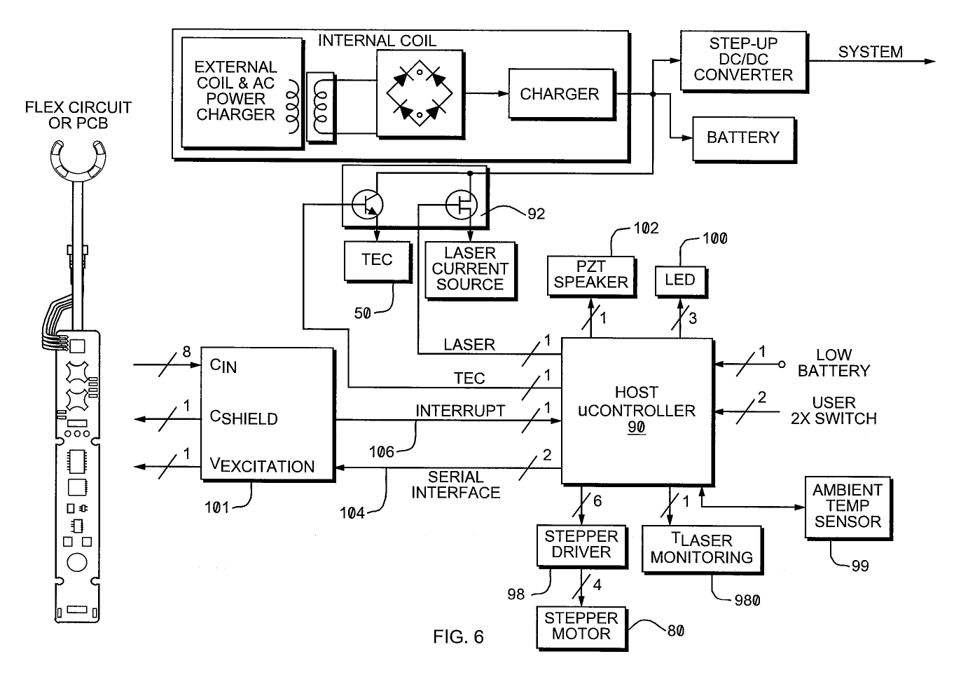

The exemplary handheld photocosmetic device 32 further includes a control/sensor module 88, implemented on a circuit board by utilizing, e.g., a host controller 90 (e.g., a microprocessor and its associated circuitry), one or more sensors, etc. The control/sensor module can control and/or monitor the operation of the device including, without limitation, distribution of power to various components, activation and deactivation of the EMR source, controlling the scanner, monitoring various operational parameters, and implementing safety protocols. By way of example, with reference to FIG. 6, the host controller (e.g., a microprocessor) 90 can provide command signals to a switch 92 (e.g., a transistor switch in this embodiment) for activating or deactivating the source (e.g., in this example, the switch can couple or decouple a current source 94 for the diode laser to power converter 96 so as to activate or deactivate the laser). The controller can also control the TEC 50 (e.g., it can switch the TEC on and off) so as to maintain the temperature of the EMR source within an acceptable range. In addition, the controller 90 can communicate with a stepper drive 98 for the stepper motor 80 to control the scan of the distal end of the optical fiber along a path (e.g., a spiral path in this case) over the skin. For example, the controller can initiate the scan by sending a signal to the driver. It can further control the rate of the scan by changing the rotational speed of the motor via application of appropriate signals to the driver. In addition, the controller can receive information from a sensor 980 for monitoring the laser's temperature and take appropriate action (e.g., deactivate the laser) if the laser's temperature begins to exceed a predefined threshold. Further, a temperature sensor 99, in communication with the controller 90, for sensing ambient temperature of the interior of the device can also be optionally provided. The controller can also effect generation of visual and auditory indicators (e.g., via an LED 100 and/or a speaker 102) to inform a user of various operational conditions of the device. The controller can also receive instructions from a user, e.g., via a serial interface 104 as well as an interrupt line 106. For example, a user can send a signal to the controller via a capacitance-to-digital (CCD) converter 101 and the interrupt line 106 to deactivate the source. Other instructions, e.g., communicated via the CCD 101 and the interface line 104, can include, e.g., a request to switch on the device or activate the EMR source to apply EMR to the skin.

In many embodiments, an optical coupler that couples EMR from the source into the optical fiber provides a high optical coupling efficiency (e.g., greater than about 80%). This advantageously allows a more efficient delivery of EMR to the skin.

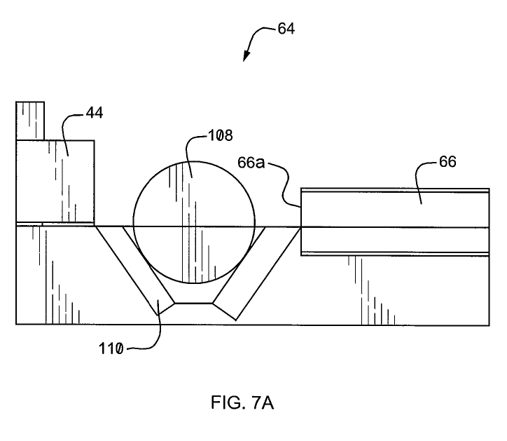

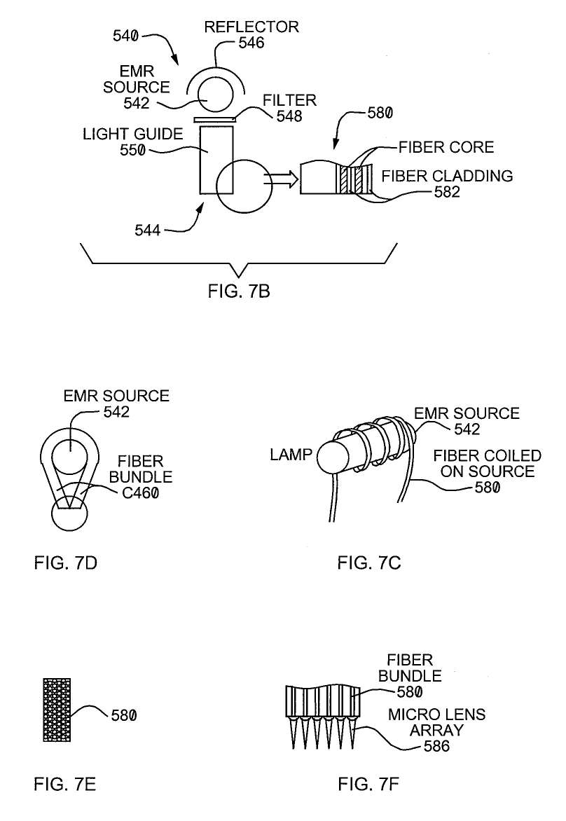

By way of example, with reference to FIG. 7A, the optical coupler 64 utilized in this exemplary embodiment includes a rod lens 108 (e.g., a fast axis rod lens) that is disposed in a V-groove 110 between the EMR source 44 and the proximal tip 66a of the optical fiber 66b. A collimating lens, such as a fast axis collimating lens (FAC) is useful to couple EMR from a source (e.g., laser diode) into at least one optical fiber (e.g., a multimode fiber). Alternatively, a pair of cylindrical lenses perpendicular to each other can collimate the highly divergent astigmatic beam coming from a laser diode. Two distinct cylindrical lenses allow complete removal of the astigmatism inherent to laser diodes through proper focusing of the lenses in each direction. Since the lens closer to the laser collimates the fast axis of the diode, this lens should have high numerical aperture (NA) to match the fast axis beam divergence. The other lens collimates the slow axis of the laser diode and therefore does not require very high NA since the light from the laser diode in the horizontal plane is less divergent.

In many implementations, the optical coupler 64 provides an optical coupling efficiency (defined as the fraction of the optical energy emitted by the source that enters the optical fiber) greater than about 80%, preferably greater than about 85%, and more preferably greater than about 90%. Such high optical coupling efficiency allows a more efficient delivery of optical energy to each discrete location of the skin, which can in turn result in an enhanced photocosmetic outcome in a shorter time. In addition, such high optical coupling efficiencies facilitate incorporating the EMR source into a handheld housing so as to provide a handheld device.

FIG. 7B shows another embodiment of the invention including an EMR source 542, an optical reflector 546, one or more optical filters 548, a light duct 550 (or concentrator), and a cooling plate (not pictured). The distal end 544 of the concentrator 550 can include an array shaped in a manner to create output light spatial modulation and concentration, and therefore to form islets of treatment in a patient's skin. For example, the distal end 544 can include an array of pyramids, cones, hemispheres, grooves, prisms, or other structures for output light spatial modulation and concentration. The distal end, therefore, can be made from any type of array, such as micro prisms, that create output modulation and concentration to produce islets of treatment.

In the embodiment of FIG. 7B, the light guide 550 can be made from a bundle of optical fibers 580 doped with ions of rear earth metals. For example, the light guide 550 can be made from a bundle of Er³⁺ doped fiber. The active ions inside the light guide core 582 can act as fluorescent (or super fluorescent) converters to provide desired spatial modulation and spectrum conversion. Thus, the light guide 550 in the embodiment of FIG. 7B can create spatial modulation of the EMR in order to create islets of treatment.

FIGS. 7C, 7D, and 7E show embodiments in which the optical fibers 580 are wrapped around the EMR source 542 in order to couple light into the optical fibers 580. As shown in FIG. 7D, each individual fiber or group of fibers 580 can have its output directed to the skin. FIG. 7E shows a bottom view of the output from the hand piece. As shown in FIG. 7E, the fibers 580 can have an output distribution that is spatially modulated in order to create islets of treatment.

FIG. 7F shows another embodiment that uses the same general structure as the embodiments of FIGS. 7B, 7C, and 7D. In the embodiment of FIG. 7F, the output of the fiber bundle 580 (i.e., the bundle of FIGS. 7C-E) can have a distal end that is made from an array of micro lenses 586 attached to the output face of the light guide. The array of micro lenses 586 can serve to focus and concentrate the output from the fiber bundle 580 in order to create islets of damage.

Referring again to FIGS. 3A and 3B, in use, the output window 86 of the handheld device 32 can be put in contact with, or in proximity of, the skin and the controller 90 can be instructed (e.g., via a signal generated when the user pushes the button 38) to cause delivery of EMR to a plurality of discrete skin locations. In some implementations, the controller 90 can selectively activate the EMR source 44 in coordination with the movement of the fiber tip over the skin, which is effectuated by the scanning mechanism in a manner discussed above, to cause delivery of EMR to a plurality of separate discrete locations along the path of the fiber tip's motion. As in this exemplary implementation, the distal end of the fiber tip follows a spiral path, the selective activation of the EMR source would result in the delivery of the EMR to a plurality of discrete locations along that path, as illustrated in FIG. 2B. In other implementations, the path traversed by the distal tip of the fiber can be different than a spiral path. For example, the fiber tip can be moved in a raster pattern over the skin and the diode laser can be selectively activated to deliver optical energy to discrete locations along the raster pattern to generate, e.g., a square grid of skin locations to which the EMR is applied as shown schematically in FIG. 2A.

The discrete locations, or optical islets can be formed in any shape which can be produced by the devices described below, limited only by the ability to control EMR beams within the tissue. Thus, depending upon the various parameters affecting the treatment, such as wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the discrete locations or islets can be variously-shaped volumes extending from the surface of the skin through one or more layers, or extending from beneath the surface of the skin through one or more layers, or within a single layer. If the beams are not convergent, such beams will define volumes of substantially constant cross-sectional areas in the plane orthogonal to the beam axis (e.g., cylinders, rectanguloids). Alternatively, the beams can be convergent, defining volumes of decreasing cross-sectional area in the plane orthogonal to the central axis of the beams (e.g., cones, pyramids). The cross-sectional areas can be regular in shape (e.g., ellipses, polygons) or can be arbitrary in shape. In addition, depending upon the wavelength(s) and fluence of an EMR beam, and the absorption and scattering characteristics of a tissue for the wavelength(s), an EMR beam may penetrate to certain depths before being initially or completely absorbed or dissipated and, therefore, an EMR-treated discrete location may not extend through the entire depth of the skin but, rather, may extend between the surface and a particular depth, or between two depths below the surface.

Generally, the lattice is a periodic structure of discrete locations or islets in one, two, or three dimensions (but can also be aperiodic). For instance, a two-dimensional (2D) lattice is periodic in two dimensions and translation invariant or non-periodic in the third. The type of periodicity is characterized by the voxel shape. For example, and without limitation, there can be layer, square, hexagonal or rectangle lattices. The lattice dimensionality can be different from that of an individual islet. A single row of equally spaced infinite cylinders is an example of the 1D lattice of 2D islets (if the cylinders are of finite length this is the 1D lattice of 3D islets). The lattice dimensionality is equal to or smaller than the dimensionality of its islets (this fact follows from the fact that the lattice cannot be periodic in the dimension where its islets are translation invariant). Hence, there exists a total of 6 lattice types with each type being an allowed combination of the islet and lattice dimensionalities. For certain applications, an “inverted” lattice can be employed, in which islets of intact tissue are separated by areas of EMR-treated tissue and the treatment area is a continuous cluster of treated tissue with non treated islands.

Each of the treated volumes can be a relatively thin disk, a relatively elongated cylinder (e.g., extending from a first depth to a second depth), or a substantially linear volume having a length which substantially exceeds its width and depth, and which is oriented substantially parallel to the skin surface. The orientation of the lines for the islets in a given application need not all be the same, and some of the lines may, for example, be at right angles to other lines. Lines also can be oriented around a treatment target for greater efficacy. For example, the lines can be perpendicular to a vessel or parallel to a wrinkle. Islets, or discrete locations, can be subsurface volumes, such as spheres, ellipsoids, cubes or rectanguloids of selected thickness. The islets can also be substantially linear or planar volumes. The shapes of the islets are determined by the combined optical parameters of the beam, including beam size, amplitude and phase distribution, the duration of application and, to a lesser extent, the wavelength.

The size of the individual islets within the lattices of EMR-treated islets of the invention, can vary widely depending upon the intended cosmetic or medical application. In some embodiments it is desirable to cause substantial tissue damage to destroy or eliminate a structure or region of tissue (e.g., a sebaceous gland a hair follicle, or tissue ablation) whereas in other embodiments it is desirable to cause little or no damage while administering an effective amount of EMR at a specified wavelength (e.g., photobiostimulation). As noted above with respect to damage islets, however, the healing of damaged tissues is more effective with smaller damage islets, for which the ratio of the wound margin to volume is greater.

The size of the EMR-treated islets of the present invention can range from 11m to 30 mm in any particular dimension. For example, and without limitation, a lattice of substantially linear islets can consist of parallel islets have a length of approximately 30 mm and a width of approximately 10 μm to 1 mm. As another example, and without limitation, for substantially cylindrical islets in which the axis of the cylinder is orthogonal to the tissue surface, the depth can be approximately 10 μm to 4 mm and the diameter can be approximately 10 μm to 1 mm. For substantially spherical or ellipsoidal islets, the diameter or major axis can be, for example, and without limitation, approximately 10 μm to 1 mm. Thus, in some embodiments, the islets can have a maximum dimension in the range from 1 μm to 10 μm, 10 μm to 100 μm, 100 μm to 1 mm, 1 mm to 10 mm, or 10 mm to 30 mm, as well as all possible ranges within 1 μm to 30 mm.

Because of the scattering effects of tissue, the minimum size of an EMR-treated islet increases with the targeted depth in the tissue, ranging from several microns on the stratum corneum to several millimeters in subcutaneous tissue. For a depth of approximately 1 mm into a subject's tissue, the minimum diameter or width of an islet is estimated to be approximately 100 μm, although much larger islets (e.g., 1-10 mm) are possible. The size of a damage islet can be either smaller or larger than the size of the corresponding optical islet, but is generally larger as greater amounts of EMR energy are applied to the optical islet due to heat diffusion. For a minimum size islet at any particular depth in the skin, the wavelength, beam size, convergence, energy and pulse width have to be optimized.

The EMR-treated islets of the invention can be located at varying points within a tissue, including surface and subsurface locations, locations at relatively limited depths, and locations spanning substantial depths. The desired depth of the islets depends upon the intended cosmetic or medical application, including the location of the targeted molecules, cells, tissues or intercellular structures.

For example, optical islets can be induced at varying depths in a tissue or organ, depending upon the depth of penetration of the EMR energy, which depends in part upon the wavelength(s) and beam size. Thus, the islets can be shallow islets that penetrate only surface layers of a tissue (e.g., 0-50 μm), deeper islets that span several layers of a tissue (e.g., 50-500 μm), or very deep, subsurface islets (e.g., 500 μm-4 mm). Using optical energy, depths of up to 25 mm can be achieved using wavelengths of 1,000-1,300 nm. Using microwave and radio frequency EMR, depths of several centimeters can be achieved. For thermal islets or damage islets, subsurface islets can be produced by targeting chromophores present only at the desired depth(s), or by cooling upper layers of a tissue while delivering EMR. For creating deep thermal or damage islets, long pulse widths coupled with surface cooling can be particularly effective.

In cases in which the EMR source provides pulses of electromagnetic radiation, the temporal separation of the pulses in conjunction with the motion of the distal tip of the optical fiber can result in applying EMR to a plurality of discrete skin locations along the path of the of the fiber tip's motion.

The use of the optical fiber advantageously results in an EMR beam for coupling into the skin that exhibits a substantially homogeneous cross-sectional intensity distribution. In particular, the EMR beam generated by the source and coupled into the fiber undergoes multiple reflections as it traverses through the fiber. These reflections substantially homogenize the cross-sectional intensity of the output beam from the fiber. In this exemplary embodiment, the optical fiber can have, e.g., an output tip with a diameter of about 100-300 microns and a NA of about 0.5 to about 4, though other tip sizes and/or numerical apertures can also be utilized. While treatment parameters can vary, in some embodiments, between about 50-200 discrete skin locations are treated per treatment site, with approximately 50-1000 discrete skin locations/cm². Further, as shown in FIG. 3F, in some implementations, a microoptic (e.g., a microlens) 1 can be coupled to the distal end of the optical fiber to providing shaping and/or focusing of the output beam. In other cases, the optical fiber can include a tapered end to impart a desired cross-sectional shape (e.g., square) to the output beam.

In some embodiments, the pitch associated with the discrete skin locations to which EMR is applied (i.e., the distance between neighboring locations) can be adjusted by regulating the speed at which the fiber's distal tip moves over the skin. For example, referring to FIGS. 3A and 3B, for a given repetition rate of the pulses generated by the EMR source, in order to increase the pitch, the controller can cause the stepper motor to rotate the gears 78 and 72 at a faster rate, thereby increasing the speed at which the fiber's distal tip moves over the skin. Alternatively, a decrease in the rotational speed of the gears can result in a smaller pitch (that is, a denser packing of the discrete skin locations).

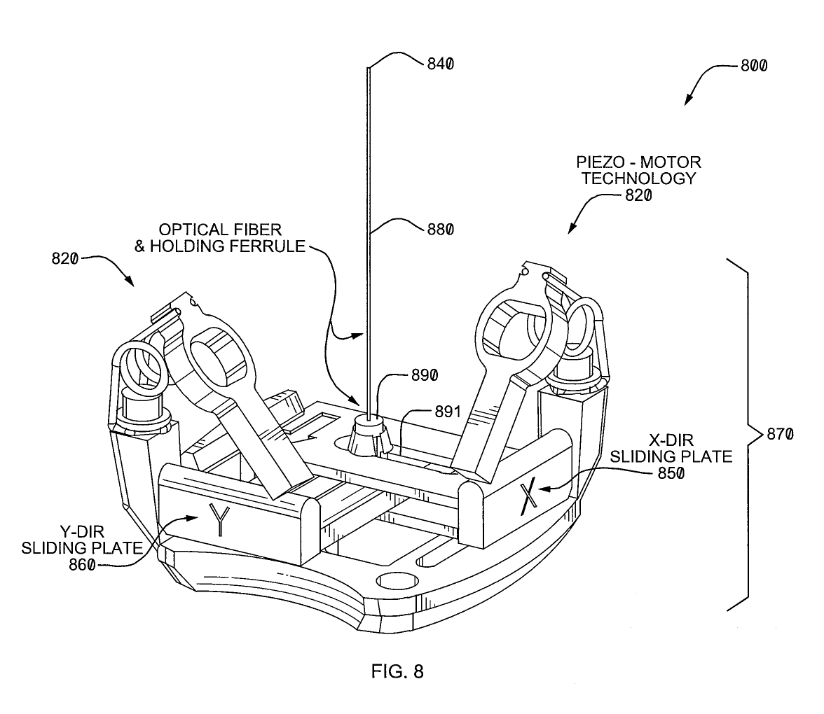

In other embodiments, piezoelectric scanning mechanisms can be employed to move the distal end of the fiber over the skin. By way of example, FIG. 8 schematically depicts an exemplary implementation of such a scanning mechanism that includes one or more motors 82 is included in the device 10 in order to move the fiber 840 in a predetermined pattern. The motor 820 can be any suitable motor, including, for example, a stepper motor, a linear motor, a piezoelectric motor, or resonant piezoelectric motor.

In one embodiment, the distal end of the fiber 840 is coupled to a fiber guide assembly system 870 so that the optical fiber 840 can be moved is a pre-determined pattern. The X-Y linear scanner 800 includes a fiber holding ferrule 880 coupled to a connector 890 that connects the fiber 840 to a fiber guide assembly system 870 comprising an x-direction sliding plate 850 and y-direction sliding plate 860 though aligned slots 891 in the plates. The ferrule 880 keeps the fibers 840 accurately aligned within the connector 890. Each sliding plate is coupled to a motor 82 so that when the motor pushes against the sliding plate, the fiber 840 moves in a horizontal (x-) and/or vertical (y-) direction. In some embodiments, the 2D movement of the fiber is coordinated with the activation of the EMR source. A variety of pre-determined spatial patterns can be programmed into the scanner and can be selected by the manufacturer, or can be selected by the user through control features on the housing (not shown). In such an embodiment, the user is able to choose from a number of different islets of treatment patterns in the skin through the use of the same hand piece. In order to use this embodiment of the invention, the user can manually place the aperture on the target area of the skin prior to firing, similar to the embodiments described earlier. In other embodiments, the aperture need not touch the skin. In such an embodiment, the device may include a stand off mechanism (not shown) for establishing a predetermined distance between the aperture and the skin surface.

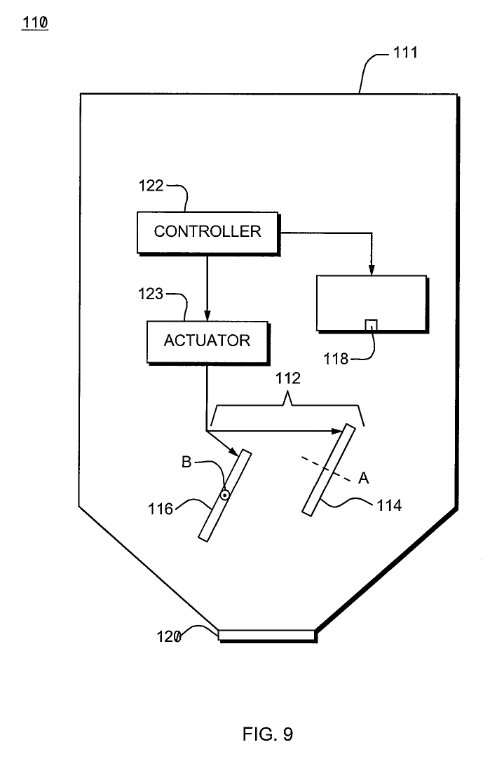

In other embodiments, the EMR delivery mechanism can include two rotating mirrors that are adapted to rotate about two orthogonal axes to scan the EMR from a source in two dimensions over the skin. By way of example, FIG. 9 schematically depicts a handheld photocosmetic device 110 that includes an EMR delivery mechanism 112 comprising two rotatable mirrors 114 and 116, which can rotate about orthogonal axes A and B. The mirror 114 can receive an EMR beam from a source 118 and transmit that EMR to the mirror 116, which can in turn direct the EMR to the skin through an EMR transmissive output window 120. The rotation of the mirrors can be utilized to scan the beam over a two-dimensional area of the skin. In some implementation, a controller 122 can synchronize the rotation of the mirrors with the emission of EMR by the EMR source to cause delivery of EMR to a plurality of separated discrete skin locations. Further details regarding scanning mechanisms utilizing rotating mirrors for applying EMR to a plurality of discrete skin locations can be found in U.S. Pat. No. 6,997,923 entitled “Method and apparatus for EMR treatment,” and co-pending U.S. application Ser. Nos. 11/097,841, 11/098,036, 11/098,015, 11/098,000, entitled “Methods and products for producing lattices of EMR-treated islets in tissues, and uses therefore” filed Apr. 1, 2005, which are hereby incorporated by reference. One advantage of a scanning system utilizing rotating mirrors is that it can more quickly scan a large area of the skin.

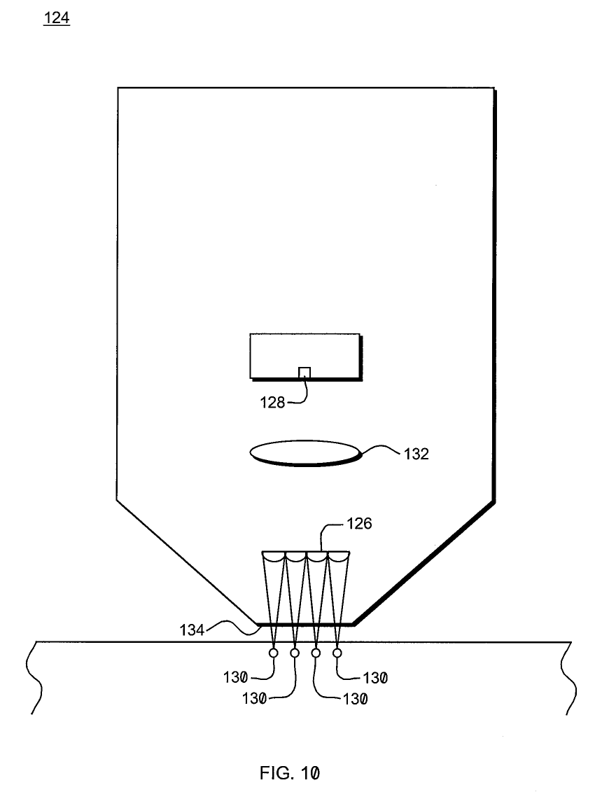

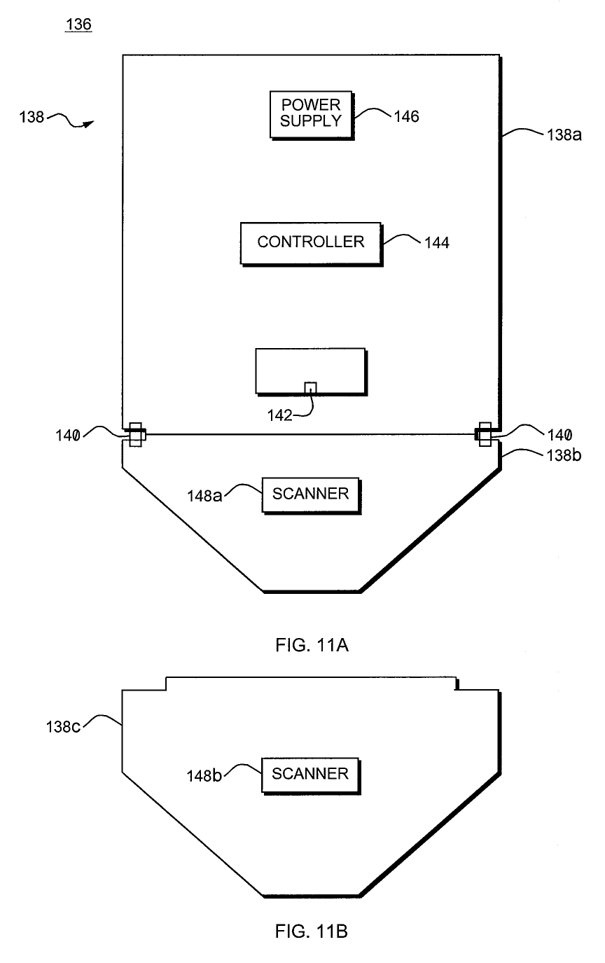

With reference to FIG. 10, in another embodiment of a handheld device 124, a plurality of microlenses 126 receive EMR generated by an EMR source 128, via a collimating lens 132, and apply that EMR as a plurality of separate EMR beams 130, through an EMR transmissive window 134, to a plurality of discrete skin locations. In some embodiments, one or more focusing elements can be disposed between the microlenses 126 and the window 134 for provide focusing of the beams. In some embodiments, the EMR transmissive window 134 can be made from a lattice of microlenses that serves to provide spatial modulation of the power density in the lattice of optical islets. Further details regarding EMR delivery systems utilizing such microlenses can be found, e.g., in U.S. Pat. No. 6,511,475 entitled “Heads for dermatology treatment” which is incorporated by reference.

In many embodiments, a variety of safety mechanisms can be incorporated in a handheld device of the invention to ensure its safe operation. For example, referring again to FIGS. 3A and 3B, the capacitance contact sensor 84 that can detect whether the distal tip of the device is within a preselected distance of the skin (e.g., a distance less than about 5 mm from the skin, or less than about 3 mm from the skin, or less than about 2 mm from the skin). The controller 90 receives the output signal of the sensor and controls the activation of the EMR source based on that signal. For example, if the sensor fails to detect an appropriate distance of the device's distal tip relative to the skin, it inhibits the activation of the EMR source or deactivates the source if it is emitting EMR. In addition, in some implementations, upon an indication from the sensor that the device is not properly positioned over the skin, the controller can activate a visual indicator (e.g., the red LED light 40C disposed on the housing) to alert the user.

Other sensors can also be incorporated in the device. For example, referring to FIG. 6, the temperature sensor 98 disposed in the housing (e.g., on the control board 88) can monitor the temperature of the EMR source. Further, other temperature sensors (e.g., the temperature sensor 99) can be incorporated in the housing to monitor the ambient temperature within the housing. The output signals of the temperature sensors can be sent to the controller, which can be programmed to provide an appropriate responses to the signals from the sensor. For example, the controller can deactivate the EMR source if the temperature indicated by the sensor is above a predefined threshold.

As yet another safety feature, in some implementations, the total optical energy applied to the skin during a treatment session (e.g., defined as a preselected time interval following the initial activation of the EMR source after the device is switched on) can be tracked to ensure that the total energy applied to the skin during the session remains below a predefined threshold. For example, the controller 90 can be programmed to calculate the total applied energy in real-time, e.g., based on the repetition rate of the pulses generated by the EMR source, the energy per pulse, the efficiency of optical coupling between the EMR source and the optical fiber that delivers the energy to the skin, and the efficiency of coupling the optical energy from the fiber into the skin. Once the total energy begins to exceed a predefined threshold, the controller can deactivate the source, and allow its reactivation only after a selected time interval has elapsed.

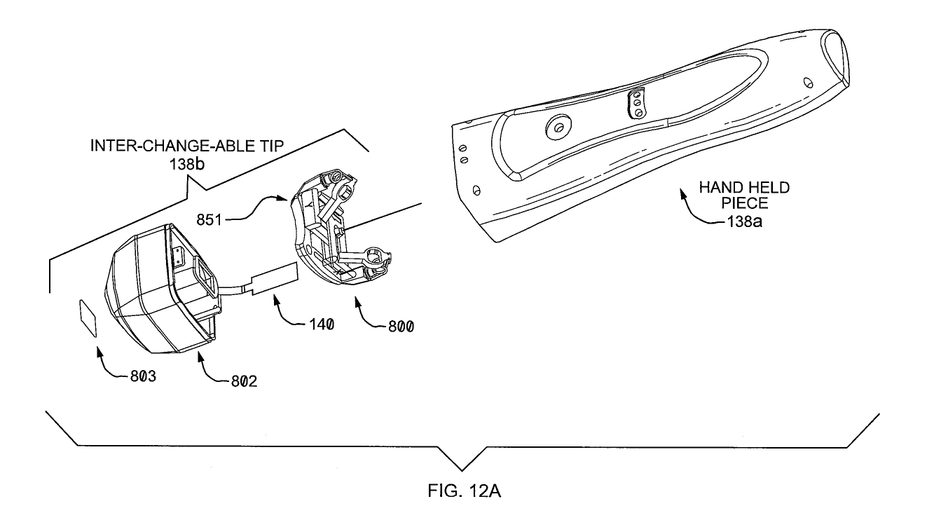

In some embodiments, the housing of the handheld device can be formed of a plurality of modular portions—each containing certain components of the device—that can be separated from one another and reconnected. By way of example, FIG. 11A schematically depicts a handheld device 136 according to such an embodiment that includes a housing 138 having two modular portions 138a and 138b, which are removably and replaceably joined via a plurality of connectors 140. In this embodiment, an EMR source 142 as well as a controller 144 and a power supply system 146 are disposed in the portion 138a and a scanning mechanism 148 for delivering the EMR emitted by the source to a plurality of discrete skin locations is disposed in the other housing portion 138b. The modularity of the device 136 advantageously allows utilizing the same EMR source and control circuitry with a variety of different scanning mechanisms. This can not only expedite the manufacturing process but lower the manufacturing cost.

Further, in some implementation, a single module having an EMR source can be provided with two or more modules containing different scanning mechanisms to allow a user to readily utilize the device for different photocosmetic applications. For example, the module 138b can be swapped with another module 138c having a different scanning mechanism 148b, shown schematically in FIG. 1l B.

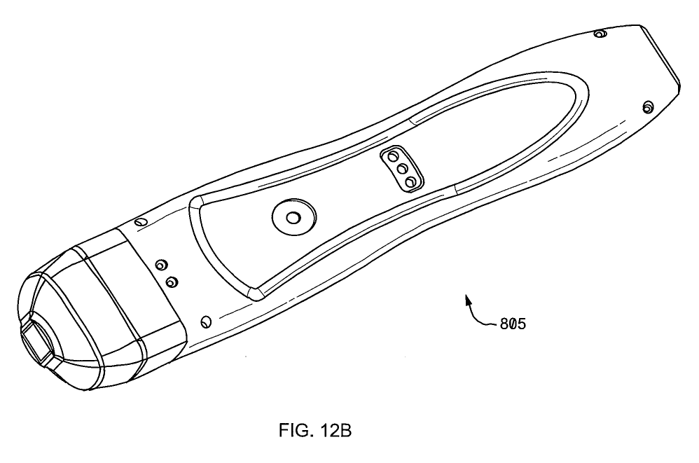

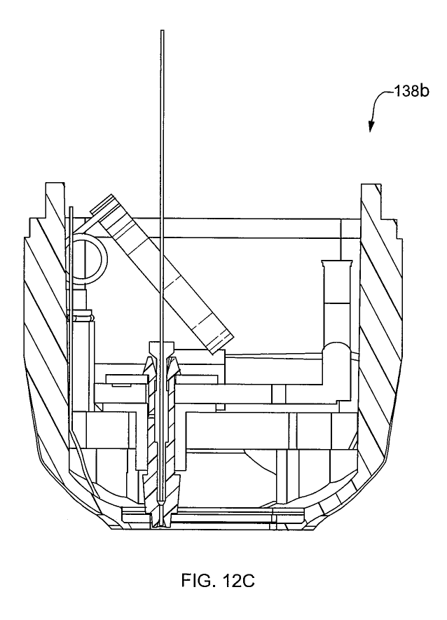

By way of example, FIG. 12A shows exemplary connectors 140 that be employed to removably and replaceably attach modular portions 138a and 138b of the housing that can be connected to form the device 805 shown in FIG. 12B. As shown in the exploded views of FIGS. 12A and 12C, modular portion 138a includes a scanner 800 that can be removeably and replaceably coupled into the tip housing 802. The type of scanner (e.g., X-Y linear scanner, spiral scanner, free beam scanner employing mirrors and/or other optical elements, etc.) inserted into the tip housing 802 can be detected by the hand piece 138a. For example, different shaped connectors 140 can be used, or an indicator 851 (e.g., bar code) can be used indicate the type of tip housing and/or scanner to the control electronics in the hand piece 138a.

The device of FIG. 12A can have an optical coating (i.e., on the treatment window 803) to provide light spatial modulation. Some embodiments can use technology similar to a gradient mirror, which is a mirror with variable transmission over its radius. An embodiment including a plurality of gradient mirrors could be beneficial for enhancement of parameters of the light source (such as the effect of photon recycling) and system cooling capabilities (very thin coating thickness).

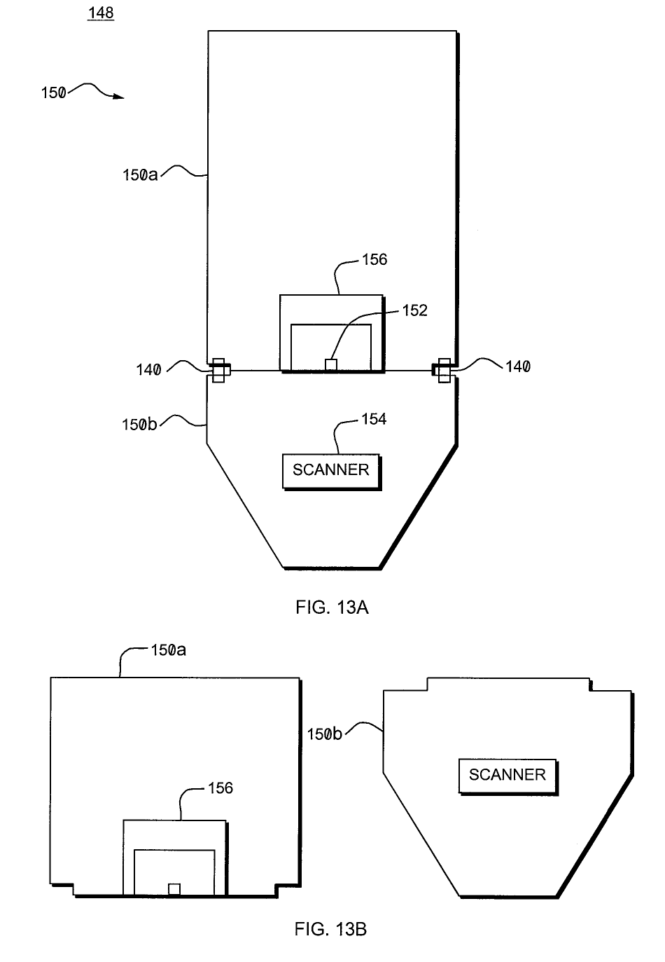

In some cases, the modularity of the device permits replacing one EMR source with another, e.g., to provide EMR in another portion of the electromagnetic spectrum or to repair the device. By way of example, FIG. 13A schematically depicts a handheld device 148 according to one such embodiment that includes a modular housing 150 having a portion 150a in which an EMR source 152 and associated control and power circuitry (not shown) are disposed, and another portion 150b (which is removably and replaceably engaged with the portion 150a, e.g., via connectors 140) in which a scanner 154 (or other light delivery mechanisms (e.g., a plurality of microlenses)) are disposed. The EMR source 152 is disposed in a removable and replaceable cartridge 156, which can be swapped with another cartridge containing a different EMR source. For example, as shown schematically in FIG. 13B, the modular portions 150a and 150b can be separated to provide access to the cartridge 156, which can be removed and replaced with another cartridge having a different EMR source (not shown). In some implementations, upon placement of a new EMR source in the housing, the controller can determine what type of source it is through the use of a detector and instruct the scanner to work in coordination with the source. For example, the controller can modify the scan pattern, pulse width, depth of focus, and/or numerical aperture. The detector system can be, for example a mechanical, optical, or electrical detector. In some embodiments, a control system recognizes and controls the various combinations of modules. For example, each module is designed to provide an identifier to the controller, which uses the identifiers to determine acceptable parameters for treatment, to restrict unacceptable parameters, and to control the operation of the device for a given combination of modules.

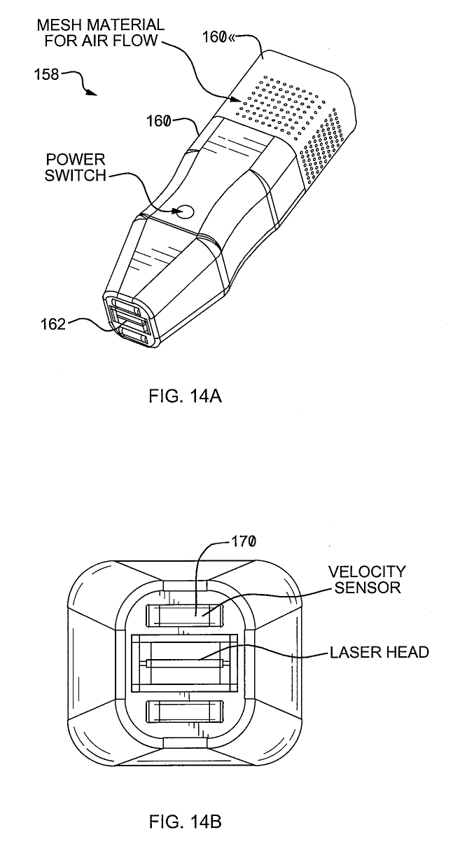

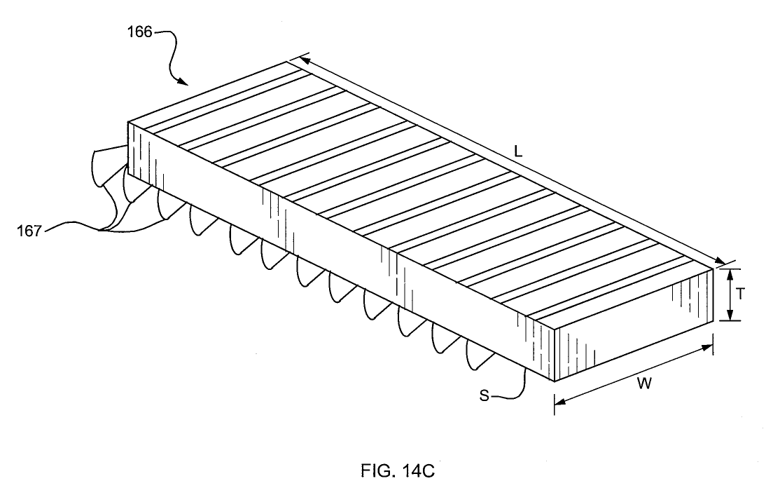

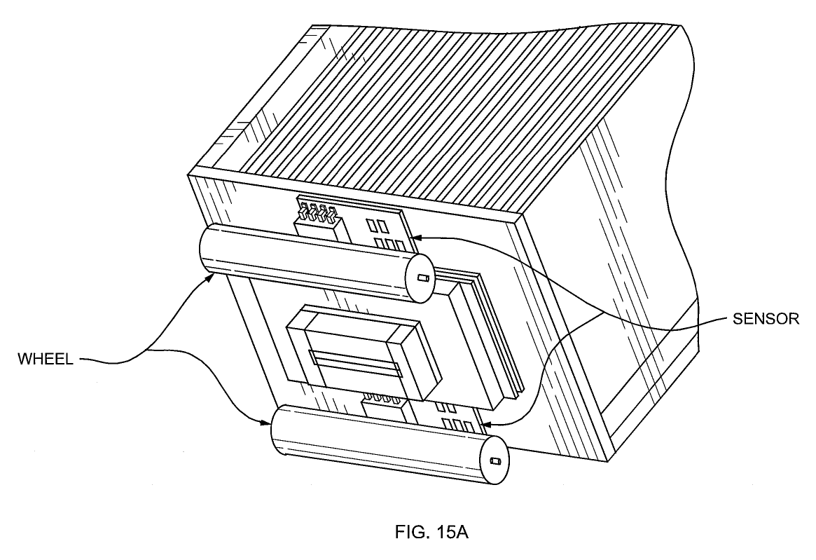

FIGS. 14A and 14B schematically depict a handheld dermatological device 158 in accordance with another embodiment that includes a housing 160 in which a plurality of EMR sources 162 are disposed. The EMR sources are thermally coupled to a cooler (not shown), e.g., such as those discussed above in connection with the previous embodiments, that extracts heat from the EMR sources to ensure that their operating temperatures remain within an acceptable range. In this implementation, the housing 160 includes a portion 160′ formed of a mesh material that allows air flow between the interior of the housing and the external environment to facilitate cooling of the device.

As shown in FIG. 14C, in this exemplary embodiment, the EMR sources comprise a diode laser bar 166 providing a plurality of EMR beams 167 for application to the skin. In a preferred embodiment, the diode laser bar 166 has length L of around 1 cm, a width W of around 10 mm, and a thickness T of around 0.0015 mm. Although in this embodiment the EMR beams have one or more wavelengths in the infrared region of the electromagnetic spectrum (e.g., in a range of about 290-10000 nm), in other embodiments the EMR beams have other wavelengths. In some implementations, one or more focusing elements (e.g., one or more lenses) can be disposed between the EMR sources and the output window to provide focusing of the EMR delivered to the skin. In this exemplary embodiment, however, the diode laser bar is placed sufficiently close to the window to obviate the need for such focusing elements.

By coupling the fiber directly into the diode bar, which is located within the device, the EMR produced is channeled directly to the surface of the skin using a flexible delivery method. Thus, the laser diode bar is not moved, optics are not required, and there is no need to precisely align optical elements. Thus, the resulting device is made more reliable, more durable, less expensive, and smaller. Further, in embodiments that have a single laser diode and moving the flexible delivery mechanism to the desired treatment locations, additional laser diodes, laser diode bars and stacks of bars are not necessary, which further decreases the cost of the device as well as the peak power requirements. Thus, by firing a single laser diode (or a few laser diodes in some alternate embodiments) repeatedly during the course of a treatment, the device is able to be operated from a lower power energy source, such as commonly available and relatively inexpensive rechargeable batteries. Further, by reducing the peak power requirements of the device, less aggressive cooling is required. Thus, the device can be cooled with, for example, a TEC or heat fin and fan rather than a chiller.

Furthermore, in embodiments where the distal end of the fiber is located at, or near, the surface of the tissue being treated, sufficient energy is transferred directly into the tissue without optics, or using relatively inexpensive optics (for example, a lens optically and/or physically coupled to the end of the fiber to focus and/or converge the EMR that is irradiated). Such a configuration also allows the device to be more robust, durable, and less expensive. Furthermore, in some embodiments, efficacy if improved, due to the direct contact and/or close proximity between the end of the fiber where the EMR is irradiated and the surface of the tissue.

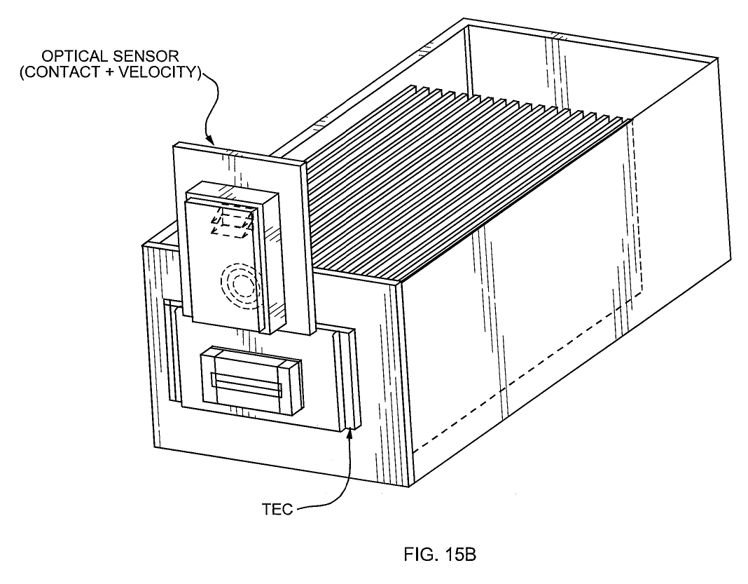

With continued reference to FIGS. 14A and 14B, the exemplary handheld device 158 can further include a velocity sensor 170 that determines the velocity of the device as it is moved over the skin. With reference to FIG. 15A, by way of example, the velocity sensor can be a mechanical sensor 171 that employs, e.g., a plurality of wheels 173 and a Hall sensor 175, to determine the velocity the device over the skin. In another example, shown schematically in FIG. 15B, an optical sensor 177 that can determine the velocity of the device directly or indirectly (e.g., by determining the rate of rotation of the wheels 173). Further details regarding velocity sensors suitable for use in the practice of the invention can be found, e.g., in co-pending U.S. application Ser. Nos. 11/097,841, 11/098,036, 11/098,015, 11/098,000, which are incorporated by reference.

Referring again to FIG. 14A, the device 158 can be employed in a stamping mode or a scanning or sliding mode. For example, in the stamping mode, the device can be placed in contact with, or in proximity of, the skin and the diode laser bar can be activated to apply each of the EMR beams to a discrete skin location. The device can then be moved to another skin portion to apply EMR thereto. In stamping modes, the resulting temperature in the skin (and, possibly, the damage profile) is determined by the geometry of the openings and the illumination/cooling parameters. In the sliding modes, an additional degree of control is available by varying the velocity of scanning.

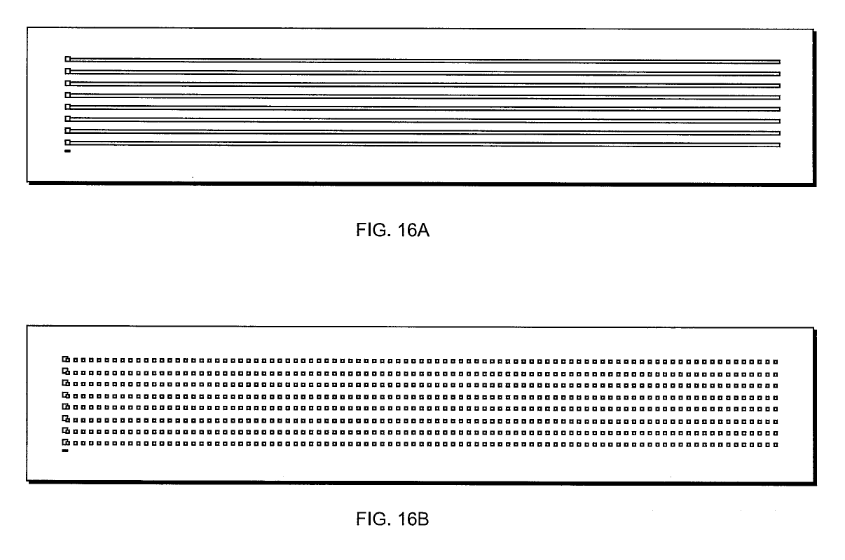

Alternatively, the device 158 can be utilized in a scanning mode. For example, the device can be scanned over a skin portion while the EMR sources are applying EMR to the skin. In some cases, where the EMR sources provide continuous EMR or pulsed EMR at a repetition rate that is considerably faster that the velocity of the device over the skin, the skin portions to which the EMR is applied can correspond to a plurality of separated linear segments, as shown in FIG. 16A. In other cases, a controller can activate the EMR sources in coordination with the motion of the device over the skin so as to apply EMR to a plurality of discrete locations, as shown in FIG. 16B. The density of the skin locations to which EMR is applied can be adjusted by selective activation of the sources based on the speed at which the device is moved over the skin, as detected by the velocity sensor 170.

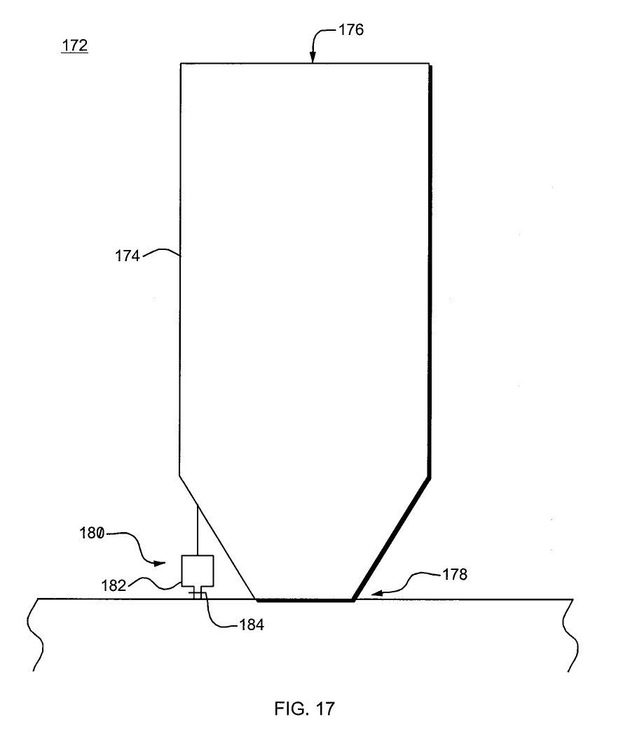

In some embodiments, a lotion dispenser can be mounted onto the handheld housing of the device to apply lotion to the surface of the skin portion to which EMR is applied. By way of example, FIG. 17 schematically depicts a handheld photocosmetic device 172 that includes a handheld housing 174 extending from a proximal end 176 to a distal end 178. Similar to the previous embodiments, the device 172 includes at least one EMR source disposed in the housing and a mechanism for delivering EMR from that source, via the device's distal end, to a plurality of discrete skin locations. A lotion dispenser 180 is mounted to the distal end of the device, which includes a reservoir 182 for storing a lotion and a lotion release mechanism 184 (e.g., an actuable valve) for releasing the lotion onto the skin. The lotion dispenser can be activated manually by a user or automatically (e.g., via an electrical signal from a controller of the device) to apply lotion to the skin surface below the device's distal end. For example, when the device is employed in a stamping mode, the lotion dispenser can be activated to apply lotion to the skin and then EMR can be applied to the skin. When the device is employed in a scanning mode, the lotion dispenser can be positioned at the distal end such that it can apply lotion to a skin portion prior to application of the EMR to that portion as the distal end of the device moves over the skin.

Both scattering and absorption are wavelength dependent. Therefore, while for shallow depths a fairly wide band of wavelengths can be utilized while still achieving a focused beam, the deeper the focus depth, the more scattering and absorption become factors, and the narrower the band of wavelengths available at which a reasonable focus can be achieved. Table 1 indicates preferred wavelength bands for various depths, although acceptable, but less than optimal, results may be possible outside these bands.

TABLE 1

| Depth of | | Numerical Aperture |
|---|---|---|
| damage, μm | Wavelength range, nm | (NA) range |
| 0-200 | 290-10000 | <3 |
| 200-300 | 400-1880 & 2050-2350 | <2 |
| 300-500 | 600-1850 & 2150-2260 | <2 |
| 500-1000 | 600-1370 & 1600-1820 | <1.5 |
| 1000-2000 | 670-1350 & 1650-1780 | <1 |
| 2000-5000 | 800-1300 | <1 |

Typically, the operational wavelength ranges from about 0.29 μm to 100 μm and the incident fluence is in the range from 1 mJ/cm² to 100 J/cm². In one example, the spectrum of the light is in the range of or around the absorption peaks for water. These include, for example, 970 nm, 1200 nm, 1470 nm, 1900 nm, 2940 nm, and/or any wavelength >1800 nm. In other examples, the spectrum is tuned close to the absorption peaks for lipids, such as 0.92 μm, 1.2 μm, 1.7 μm, and/or 2.3 μm, and wavelengths like 3.4 μm, and longer or absorption peaks for proteins, such as keratin, or other endogenous tissue chromophores contained in the tissue.

The wavelength can also be selected from the range in which this absorption coefficient is higher than 1 cm⁻¹, such as higher than about 10 cm¹. Typically, the wavelength ranges from about 0.29 μm to 100 μm and the incident fluence is in the range from 1 mJ/cm² to 1000 J/cm². The effective heating pulse width is preferably less than 100× thermal relaxation time of the targeted chromophores (e.g., from 100 fsec to 1 sec).

Normally the pulse width of the applied EMR should be less than the thermal relaxation time (TRT) of each of the discrete locations or optical islets, since a longer duration may result in heat migrating beyond the boundaries of these portions. Since the discrete locations will generally be relatively small, pulse durations will also be relatively short. However, as depth increases, and the spot sizes thus also increase, maximum pulse width or duration also increase. The pulse-widths can be longer than the thermal relaxation time of the discrete locations if density of the targets is not too high, so that the combined heat from the target areas at any point outside these areas is well below the damage threshold for tissue at such point. Generally, thermal diffusion theory indicates that pulse width r for a spherical islet should be τ<500 D²/24 and the pulse width for a cylindrical islet with a diameter D is τ<50 D²/16, where D is the characteristic size of the target. Further, the pulse-widths can sometimes be longer than the thermal relaxation time of the discrete locations if density of the targets is not too high, so that the combined heat from the target areas at any point outside these areas is well below the damage threshold for tissue at such point. Also, with a suitable cooling regimen, the above limitation may not apply, and pulse durations in excess of the thermal relaxation time for a discrete locations, sometimes substantially in excess of TRT, may be utilized.

The required power from the EMR source depends on the desired therapeutic effect, increasing with increasing depth and cooling and with decreasing absorption due to wavelength. The power also decreases with increasing pulse width. Some embodiments of the invention use one or more diode lasers as the EMR source. Because many photodermatology applications require a high-power light source, a standard 40-W, 1-cm-long, cw diode lasers can be used in some embodiments. Any suitable diode laser bar can be used including, for example, 10-100 W diode laser bars. A number of types of diode lasers, such as those set forth above, can be used within the scope of the invention. Other sources (e.g., LEDs and diode lasers with SHG) can be substituted for the diode laser bar with suitable modifications to the optical and mechanical sub-systems.

Various light based devices can be used to deliver the required light doses to a body. The optical radiation source(s) utilized may provide a power density at the user's skin surface of from approximately 1 mwatt/cm² to approximately 100 watts/cm², with a range of 10 mwatts/cm² to 10 watts/cm² being preferred. The power density employed will be such that a significant therapeutic effect can be achieved, as indicated above, by relatively frequent treatments over an extended time period. The power density will also vary as a function of a number of factors including, but not limited to, the condition being treated, the wavelength or wavelengths employed and the body location where treatment is desired, i.e., the depth of treatment, the user's skin type, etc. A suitable source may, for example, provide a power of approximately 1-100 watts, preferably 2-10 W, designed to irradiate tissue 0.2-1 mm beneath the skin surface at a power density of approximately 0.01-10 W/cm² at the skin surface. In another aspect of the invention, the treatment can cause resolution or improvement in appearance of acne lesion indirectly, through absorption of light by blood and other endogenous tissue chromophores.

In some embodiments, a single EMR source (e.g., laser diode) will be translated to create lattices of optical islets. Lattices of optical islets generate lattices of mirco denatured zones in the skin, which promotes removal of abnormally pigmented cells and stimulates new collagen growth and can result in reduction of visibility of pigmentation spots and improvement in skin appearance and skin texture. The fractional nature of the method is less painful and heals faster than other light-based dermatology treatments.

Alternative embodiments can employ an optical delivery system that include, for example, a set of lenses to image the EMR that is generated by the source and deliver the imaged EMR to the tissue. Some such alternative embodiments could additionally include a zoom lens system as described in detail in co-pending U.S. patent application Ser. No. 11/701,192 filed Feb. 1, 2007 entitled “Dermatological Device Having a Zoom Lens System,” which is hereby incorporated by reference. The zoom lens can focus the beamlets into a plurality of skin portions (herein also referred to as islets or EMR-treated islets) separated from one another by untreated (or less treated, or differently treated) skin, as skin portions. The zoom lens allows adjustment of the pitch of the islets (distance between the islets) by changing the magnification of the image of the optical mask that it forms, and hence adjusting the density of the islets formed within the skin. The adjustment of the pitch of the focused spots can be advantageously utilized to optimize treatment of the skin for a variety of skin types and conditions, as discussed further below.

Methods of Use

In some aspects, methods and devices or provided that are appropriate for use in multi-session diode-laser fractional treatment which can be used, for example, for skin rejuvenation, wrinkle reduction, reduction of skin dyschromia, ablation of tissue, the formation of micro-holes, and other treatments.

For example, devices such as the device of FIG. 3A can be used as part of a novel periodic treatment regime. Treatments using existing fractional devices are available to a consumer through professionals, such as dermatologists or professional spas. These treatments by nature are performed using devices having very high power and relatively higher density of beams. In other words, the pitch between individual treatment islets created in tissue by a set of beams (or a single beam in the case of some devices using a scanner) is relatively small, and a relatively large number of islets per unit of area and/or volume of tissue are created. This provides for a more intense treatment, and is designed to improve the efficacy of the single treatment. In other words, professional devices are designed to treat as much tissue as possible in a single treatment in order to obtain results in only one or a few treatments.

However, the inventors have discovered that better results can be obtained by treating the tissue less intensely, but more frequently. For example, the device 32 of FIG. 3A produces islets in the tissue that are relatively less dense than those produced by professional devices. In other words, the pitch between the islets is greater than in existing professional devices. Similarly, the power density applied per islet is lower than in a typical professional treatment. Thus, in a single treatment, fewer islets are created per unit of area and/or volume of tissue than in a typical professional treatment, and a single treatment using the device will typically result in less tissue damage. While such a single treatment will not be as efficacious as a single treatment using a professional device, producing less damage in a single treatment allows the user to safely perform subsequent treatments much sooner without excessively damaging the tissue. By providing a device that is easily accessible, e.g., used in the home, the subject can more easily and regularly perform such treatments, which are impractical in the professional or medical setting do to the logistical difficulty and cost to the typical subject of frequently attending appointments with a professional provider.

In initial clinical testing of devices similar to the device 32 of FIG. 3A, the inventors have discovered that regular and repeated application of EMR using a fractional device having less intensity per treatment than existing professional devices will result in greater efficacy over time. For example, subjects that have used devices similar to the device 32 to treat area of the face have obtained on average superior results to those seen with a typical professional treatment. An exemplary treatment protocol for skin rejuvenation is provided in Table 2.

TABLE 2
Exemplary Treatment Protocol for Skin Rejuvenation

| | Example 1 | Example 2 |
|---|---|---|
| Energy per Spot: | 5 mJ | 7 mJ |
| Density of Spots per pass: | 200/cm² | 500/cm² |
| Number of passes per session: | 5 | 2 |
| Number of treatment sessions: | 15 | 8-10 |
| Treatment Interval (days): | 2-3 | 1-3 |
| Total Cumulative Spot Density: | 15,000 | 8000-10,000 |

Subjects that used the device every other day to perform skin rejuvenation of facial tissue achieved superior results over the course of several months than are typically achieved in a series of professional treatment. Without limiting the scope of the invention, the inventors believe that this is due to the fact that the healing response of tissue responds better to gradual applications of EMR using relatively larger pitch (relatively lower islet density) that is performed frequently and repeatedly. Also without limiting the scope of the invention, the inventors also believe that repeated low intensity treatments help to maintain prior results. Also without limiting the invention, the inventors believe that the more gradual treatment over time allows for a greater total density of treatment spots per unit of treated area and/or volume than is possible with existing professional treatments. Based on the initial testing of various treatment protocols, the inventors expect that other treatments (such as wrinkle removal, the treatment of acne, etc.) will similarly be more efficacious when performed more frequently using less intense treatments.

Therefore, many new treatment regimes are possible. For example, a subject can be treated by a professional to receive a more intense initial treatment while subsequent less intense treatments can be performed by the subject using various embodiments of the invention. The follow up treatments could be performed using a device available over the counter or using a prescription device or other device supplied by the professional that performed the treatment. Similarly, the subject can use embodiments of the invention to perform a series of relatively low intensity treatment periodically over time (such as every other day, weekly, etc., and for a period of weeks, months or years). The subject can also use embodiments of the invention to perform an initial treatment that is more intense (for example, has relatively less pitch between islets and/or applies more energy per islet during the treatment) followed by a series of periodic follow-up treatments using parameters to achieve a less intense treatment.

Although such periodic treatments preferably employ a series of low intensity treatments on a frequent and sustained basis, many other embodiments are possible. For example, some treatments may benefit from a series of treatments performed using relatively more intense parameters, such as the parameters typically employed in professional treatments. Similarly, the device may be used with the same frequency as a professional treatment.

Additional Photocosmetic Applications

Many additional applications are possible. For example, devices similar to those described herein may be used to perform fractional ablation and the formation of micro holes. Additional detailed disclosure of this application if provided in U.S. Provisional Patent Application 60/877,826 entitled “Methods And Products For Ablating Tissue Using Lattices Of EMR-Treated Islets”, which is currently pending and which is incorporated herein by reference.

Non-ablative applications include the selective treatment of structures within the skin, such as pigmented lesions, vascular lesions and vein treatments. These and other similar application are described in greater detail in U.S. Provisional Application 60/923,093 entitled “Photoselective Islets In Skin and Other Tissues” which is currently pending and which is incorporated herein by reference.

Treatment of the dermis, especially the deep layers of dermis are also possible. These and other similar application are described in greater detail in U.S. Provisional Application 60/923,398 entitled “Deep Fractional Thermal Treatment at Dermal/Hypodermal Junction” which is currently pending and which is incorporated herein by reference.

Embodiments of the handheld photocosmetic device can be used in a variety of additional applications in a variety of different organs and tissues. For example, treatments can be applied to tissues including, but not limited to, skin, mucosal tissues (e.g., oral mucosa, gastrointestinal mucosa), ophthalmic tissues (e.g., conjuctiva, cornea, retina), and glandular tissues (e.g., lacrimal, prostate glands). As a general matter, the methods can be used to treat conditions including, but not limited to, lesions (e.g., sores, ulcers), acne, rosacea, undesired hair, undesired blood vessels, hyperplastic growths (e.g., tumors, polyps, benign prostatic hyperplasia), hypertrophic growths (e.g., benign prostatic hypertrophy), neovascularization (e.g., tumor-associated angiogenesis), arterial or venous malformations (e.g., hemangiomas, nevus flammeus), and undesired pigmentation (e.g., pigmented birthmarks, tattoos).

In some aspects, the invention provides methods of treating tissues by creating lattices of thermal islets. These methods can be used in, for example, methods of increasing the permeability of the stratum corneum to various agents, including therapeutic agents and cosmetic agents, and methods for producing therapeutic hyperthermia.

In one embodiment, lattices of thermal islets are produced in order to reversibly increase the permeability of the stratum corneum by heating islets of tissue to temperatures of 35-100° C. The increased permeability results from the melting of the extracellular matrix of crystalline lipids that surrounds the cells of the stratum corneum and, when present, the stratum lucidum. When this matrix melts (i.e., loses its crystalline structure), the SC becomes more permeable to molecules on the surface of the skin, allowing some molecules to diffuse inward. When the temperature of the layer returns to the normal range (i.e., 29-37° C.), the intercellular matrix recrystallizes, the SC becomes more impermeable, and any molecules which had diffused below the SC can remain there, further diffuse into surrounding tissues, or enter the systemic circulation. Thus, as used herein, the increased permeability is “reversible” because the lipid intercellular matrix recrystallizes. In different embodiments, the increase in permeability is reversed within 1 second to 2 hours after the treatment is discontinued. Thus, in some embodiments, the increase in permeability is reversed within 15 minutes, 30 minutes, 1 hour or 2 hours after the EMR-treatment is discontinued.

In these embodiments, the thermal islets define permeation pathways which can extend through or mostly through the stratum corneum and stratum lucidum layers so that a compound, for example, a cosmetic or therapeutic agent applied to the exterior surface of the skin is able to efficiently penetrate the stratum corneum/stratum lucidum. This penetration can be superficial and remain just below or within the stratum corneum, or can be deeper into the interior layers of the epidermis or dermis and, possibly, into the blood stream via the vascularization in the dermis. This enables the percutaneous delivery of cosmetic or therapeutic agents locally to the epidermis and dermis. To the extent the compound diffuses away from the site of treatment, the local delivery of the compound can be greater (e.g., delivery to a joint region). Moreover, to the extent that the compound reaches the vasculature of the dermis, delivery can be systemic.

In some embodiments, the compound is a therapeutic agent. Examples of therapeutic agents include, without limitation, a hormone, a steroid, a non-steroidal anti-inflammatory drug, an anti-neoplastic agent, an antihistamine and an anesthetic agent. Specific examples include, without limitation, hormones such as insulin and estrogen, steroids such as prednisolone and loteprednol, non-steroidal anti-inflammatory drugs such as ketorolac and diclofenac, anti-neoplastic agents such as methotrexate, and antihistamines such as histamine H1 antagonists, chlorpheniramine, pyrilamine, mepyramine, emedastine, levocabastine and lidocaine.

In other embodiments, the compound is a cosmetic agent. Examples of cosmetic agents include, without limitation, pigments (including both naturally occurring and synthetic chromophores, dyes, colorants or inks) reflective agents (including light-scattering compounds), and photoprotectants (including sunscreens). Such cosmetic agents can be used to add coloration to the skin, or to mask existing coloration (e.g., birthmarks, pigmented lesions, tattoos) by adding differently colored pigments or reflective agents. The invention provides improved methods of applying cosmetic agents because (a) the agents are contained within the stratum corneum and will not be smeared, or rubbed or washed off, and (b) the agents will remain within the stratum corneum until the cells of that layer are replaced through the normal process of outgrowth from the stratum basale (e.g., approximately 21-28 days). Thus, a single application of a cosmetic agent can last for several weeks, which can be advantageous relative to cosmetics which must be applied daily. Conversely, the application of the cosmetic agent is limited to several weeks, which can be advantageous relative to tattoos which are usually permanent unless removed by photobleaching or tissue ablation. In one embodiment, pigments for a desired temporary tattoo can be applied to the skin (e.g., by a film, brush, printing), the stratum corneum can be EMR-treated to increase permeability, and the pigments can diffuse into the skin to create the temporary tattoo. In other embodiments, an artificial tan can be created by delivering a colorant or, conversely, a tan can be prevented by delivering a sunscreen into the skin.

The increased permeability of the stratum corneum can be made painless or less painful for a subject by using lattices of thermal islets (or damage islets) rather than a continuous area of heating. Because the entire area and thickness of the skin is not heated, a 40-43° C. isotherm can be terminated near the epidermis/dermis boundary instead of deeper in the dermis. Therefore, nerve endings found in papillary dermis are not exposed to the 40-43° C. temperatures associated with a pain response. As a result, the enhanced permeability paths defined by the thermal islets can be created without pain even though the SC has been exposed to temperatures significantly higher than 40-43° C.

In another aspect, the invention can involve creating many zones of increased permeability in the stratum corneum (SC) without causing irreversible structural damage, or minimizing such damage, to the tissue. Reversible permeability is achieved by creating permeability of a topical in the SC for a limited time. Generally, this limited time corresponds to the application of EMR energy. After application of the EMR energy, the SC closes. Alternatively, permeability can remain for a period of time after application of the EMR energy. The time for permeability should be achieved in a limited time to prevent risk of infection. Using the principles of the present invention, such treatment can be made safe and painless, and thus can be practiced, for example, by members of general public, i.e., individuals with no special training. One such use is for enhancing the delivery of topical cosmetic compositions or pharmaceutical agents during in-home application.

In accordance with the present invention, and as more fully described below, thermal islets can be produced which span from a tissue surface to deeper layers of the tissue, or which are present entirely in subsurface layers. Such thermal islets can be used for applications such as thermally-enhanced photobiomodulation, photobiostimulation and photobiosuspension, as well as the creation of damage islets, as described below.

In some aspects, the invention provides methods of treating tissues by creating lattices of damage islets. These methods can be used in, for example, skin rejuvenation, tattoo removal (e.g., killing cells containing ink particles, ablation of tattoo ink particles), acne treatment (e.g., damaging or destroying sebaceous glands, killing bacteria, reducing inflammation), pigmented lesion treatment, vascular lesion treatment, and nevus flammeus (“port wine stain”) removal (e.g., reducing pathological vasculature), among others. Lattices of damage islets can also be used to increase the permeability of the stratum corneum. The time for recovery or healing of such damage islets can be controlled by changing the size of the damage islets and the fill factor of the lattice.

In some embodiments, the invention provides methods of tissue remodeling based on controlled tissue damage. One embodiment of tissue remodeling is skin “rejuvenation,” a complex process involving one or more of (a) reduction in skin dyschromia (i.e., pigment non-uniformities), (b) reduction in telangiectasia (i.e., vascular malformations), (c) improvement in skin texture (e.g., reduction of rhytides and wrinkles, skin smoothing, pore size reduction), and (d) improvement in skin tensile properties (e.g., increase in elasticity, lifting, tightening). Techniques used for skin rejuvenation can be divided into three broad classes: ablative, non-ablative and fractional (including the lattices of islets of the present invention).

In the ablative resurfacing approach, the full thickness of the epidermis and a portion of upper dermis are ablated and/or coagulated. The ablative techniques typically deliver more pronounced clinical results, but entail considerable post-operative recovery time and care, discomfort, and risk of infection. For example, laser skin resurfacing (e.g., using a CO₂ laser an with absorption coefficient of about 900 cm⁻¹, or an Er:YAG laser with an absorption coefficient of about 13,000 cm⁻¹) requires weeks of recovery time, followed by a period of up to several months during which the treated skin is erythematous.

In the non-ablative approach, the zone of coagulation is shifted deeper into the tissue, with the epidermis being left intact (e.g., using lasers with absorption coefficients of 5-25 cm⁻¹). The non-ablative techniques entail considerably less post-operative recovery time and care, discomfort, and risk of infection.

The fractional approach is also non-ablative but, instead of coagulating the entire treatment area or damage zone, entails partial or fractional damage of the treatment area. That is, a lattice of damage islets is created within the treatment area.

The present invention provides methods of skin rejuvenation in which thermal and damage islets can be relatively deep in the dermis and hypodermis (e.g., depths >500 μm from the skin surface). In order to prevent epidermal damage, active or passive cooling of the epidermis can be employed.

The creation of lattices of damage islets can result in skin lifting or tightening as a result of (a) shrinkage of collagen fibrils subjected to elevated temperatures (immediate effect) or (b) coagulation of localized areas in the dermis and hypodermis (immediate to short-term effect).

The creation of lattices of damage islets can result in smoother skin texture as a result of coagulation of localized areas in the dermis and hypodermis (immediate to short-term effect). This technique also can be used for texturing tissues or organs other than the dermis/epidermis (e.g., lip augmentation).

The creation of lattices of damage islets can result in the promotion of collagen production as a result of the healing response of tissues to thermal stress or thermal shock (medium- to long-term effect). The creation of lattices of damage islets can also result in the promotion of production of hyaluronic acid as a result of the healing response of tissues to thermal stress or thermal shock (short- to medium-term effect). Repeating treatments in regular intervals can maintain the level of hyaluronic acid and as a result maintain improved skin appearance.

The creation of lattices of damage islets can be used to remove tattoos by killing the cells containing the tattoo ink particles (typically cells of the upper dermis). After these cells are killed, the tattoo ink is cleared away from the tissue site by normal scavenging processes. Alternatively, or in addition, lattices of damage islets can be used to remove tattoos by selecting the wavelength(s) of the EMR treatment to cause selective absorption of the EMR energy by the tattoo ink particles. In some embodiments, the pulse width of the incident pulse is chosen to match the thermal relaxation time of the ink particles. The absorption of the EMR energy by the tattoo ink particles can cause the cells to be heated and killed; can cause the ink particles to undergo photobleaching or be broken into smaller molecules which are removed by normal processes; or can otherwise cause the ink to be destroyed.

The creation of lattices of damage islets can be used in order to increase the permeability of the stratum corneum by heating islets of tissue to temperatures higher than 100° C. to create small holes in SC. Thus, in these embodiments, the EMR treatment coagulates, ablates, vaporizes, or otherwise damages or removes portions of the SC, including the crystalline intercellular lipid structure or cells, to form a lattice of damage islets through the SC. This method increases the permeability of the SC for a longer period of time than the thermal islet methods described above because the damaged areas or holes can remain in the SC until that layer of cells is replaced through the normal process of outgrowth from the stratum basale (e.g., approximately 21-28 days).

The creation of lattices of damage islets can be used to treat acne by selecting the wavelength(s) of the EMR treatment to cause selective absorption of the EMR energy by sebum, or targeting the lattice to sebaceous glands, in order to selectively damage or destroy the sebaceous glands. The EMR treatment can also be targeted to bacteria within acne sores.

The creation of lattices of damage islets can be used to treat hypertrophic scars by inducing shrinkage and tightening of the scar tissue, and replacement of abnormal connective tissue with normal connective tissue.

The creation of lattices of damage islets can be used to treat body odor by selectively targeting eccrine glands, thereby reducing the production of eccrine sweat or altering its composition.

The creation of lattices of damage islets can be used to treat warts and calluses by selectively targeting the pathological tissue to kill cells or cause tissue peeling. The pathological tissue can be replaced with normal tissue by normal biological processes.

The creation of lattices of damage islets can be used to treat psoriasis by using EMR of appropriate wavelength to selectively target psoriasis plaques, thereby stopping or reversing plaque formation. The pathological tissue can be replaced with normal tissue by normal biological processes.

The creation of lattices of damage islets can be used to decrease the time needed for the healing of wounds or burns (including frostbite) by increasing the wound or burn margin without substantially increasing the volume.

The creation of lattices of damage islets can be used to reduce cellulite by changing the mechanical stress distribution at the dermis/hypodermis border. Alternatively, or in addition, lattices of damage islets can be used to reduce fat in the hypodermis (subcutaneous tissue) by heating and damaging fatty cells inside islets.

The creation of lattices of damage islets can be used in order to decrease the amount or presence of body hair by targeting lattices of damage islets to hair follicles in the skin. The methods can selectively target melanin or other chromophores present in hair or hair follicles, or may non-selectively target water in the hair follicle.

The creation of lattices of damage islets can be used in order to damage or destroy internal epithelia to treat conditions such a benign prostatic hyperplasia or hypertrophy, or restenosis. The methods can also be used to weld tissues together by creating damage areas at tissue interfaces.

The creation of lattices of damage islets can be used in order to create identification patterns in tissues which result from the ablation of tissue or other structures, or which result from the tissue healing process. For example, patterns can be created in hair shafts by “etching” the hair with a lattice of damage islets. Alternatively, dermal, epidermal or other epithelial tissues can be patterned using the healing process to create defined areas with altered appearances.

In some aspects, the invention provides methods of treating tissues by creating lattices of photochemical islets. These methods can be used in, for example, activating EMR-dependent biological responses (e.g., melanin production or “tanning”) and photodynamic therapy (e.g., psoralen therapy for vitiligo or hypopigmentation). For example, vitiligo, white stretch marks (i.e., striae alba), and hypo-pigmentation can be treated by creating photochemical islets which, with or without photodynamic agents, increase the production of pigmentation in the treated areas. In particular, by targeting the stratum basale, proliferation and differentiation of melanocytes can be promoted.

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

### REFERENCES AND DEFINITIONS

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

Numerical Ranges.

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

As used herein, EMR includes the range of wavelengths approximately between 200 nm and 10 mm. Optical radiation, i.e., EMR in the spectrum having wavelengths in the range between approximately 200 nm and 100 μm, is preferably employed in the embodiments described above, but, also as discussed above, many other wavelengths of energy can be used alone or in combination. The term “narrow-band” refers to the electromagnetic radiation spectrum, having a single peak or multiple peaks with FWHM (full width at half maximum) of each peak typically not exceeding 10% of the central wavelength of the respective peak. The actual spectrum may also include broad-band components, either providing additional treatment benefits or having no effect on treatment. Additionally, the term optical (when used in a term other than term “optical radiation”) applies to the entire EMR spectrum. For example, as used herein, the term “optical path” is a path suitable for EMR radiation other than “optical radiation.”

It should be noted, however, that other energy may be used to for treatment islets in similar fashion. For example, non-EMR sources such as ultrasound, photo-acoustic and other sources of energy may also be used to form treatment islets. Thus, although the embodiments described herein are described with regard to the use of EMR to form the islets, other forms of energy to form the islets are within the scope of the invention and the claims.

1. A handheld photocosmetic device for performing fractional treatment of tissue by a user, comprising
a housing,
an EMR source disposed in the housing, and
an EMR delivery path within the housing and optically coupled to the light source,
wherein the EMR delivery path is configured to apply EMR generated by the EMR source to a plurality of discrete locations located within a treatment area of the tissue and wherein a total area of the plurality of discrete locations is less than the treatment area; and
wherein the device is configured to be self-contained within or about the housing such that substantially the entire device can be handheld by the user during operation.

2. The device of claim 1, wherein the total area of the plurality of discrete locations is between approximately 1 and 90 percent of the treatment area.

3. The device of claim 1, further comprising an electrical cord in electrical communication with the EMR source and configured to supply power to the EMR source.

4. The device of claim 1, further comprising a power source coupled to the housing and in electrical communication with the EMR source, wherein the power source is configured to supply power to the EMR source.

5. The device of claim 4, wherein the power source includes a battery.

6. The device of claim 1, wherein the discrete locations are distributed according to a predetermined or random pattern.

7. The device of claim 1, wherein the EMR delivery path comprises an optical scanner.

8. The device of claim 7, wherein the scanner comprises at least one optical fiber having an input port adapted to receive EMR from the EMR source and having an output port through which EMR can be delivered to the locations.

9. The device of claim 8, wherein the scanner further comprises a scanning mechanism coupled to the output port of the fiber for moving the output port to direct EMR to the locations.

10. The device of claim 9, wherein the scanning mechanism is optically coupled to the output port of the fiber, and further comprises one or more rotatable mirrors for directing the EMR to the locations.

11. The device of claim 9, wherein the scanning mechanism comprises at least one piezoelectric scanner element.

12. The device of claim 11, wherein the piezoelectric scanner element is an adjustable multilayer piezoelectric device.

13. The device of claim 8, further comprising optics coupled to the output port for shaping the EMR passed through the output port.

14. The device of claim 8, further comprising a controller for controlling the EMR source in substantial synchrony with the movement of the fiber's output port to effect delivery of EMR to the locations.

15. The device of claim 14, wherein the controller selectively activates the EMR source.

16. The device of claim 15, wherein the controller selectively blocks EMR emitted from the source from entry into the fiber.

17. The device of claim 8, further comprising an optical coupler disposed between the EMR source and the optical fiber for directing light from the source into the fiber.

18. The device of claim 17, wherein the coupler comprises one or more focusing optical elements for focusing EMR from the source into the fiber.

19. The device of claim 18, where the one or more focusing elements focus the EMR into the fiber at a numerical aperture in a range of about 0.5 to about 3.

20. The device of claim 8, wherein the EMR source and the input port of the optical fiber are aligned such that at least 80% of EMR energy generated by the source is coupled into the optical fiber.

21. The device of claim 17, wherein the coupler comprises a connector for selectively connecting a selected EMR source and a selected optical fiber.

22. The device of claim 1, further comprising a safety system having one or more sensors for sensing one or more operating parameters of the device.

23. The device of claim 22, wherein at least one of the sensors comprises a contact sensor for sensing contact between an EMR-emitting end of the device and the skin.

24. The device of claim 23, wherein the safety mechanism inhibits delivery of light to the skin if the contact sensor senses a contact value below a minimum contact threshold.

25. The device of claim 23, wherein the minimum contact threshold is a contact area greater than about 70% of an area of the EMR-emitting end.

26. The device of claim 23, wherein the contact sensor is selected from the group comprising conductance sensors, piezoelectric sensors, and mechanical sensors.

27. The device of claim 22, wherein the safety system inhibits delivery of EMR energy exceeding a predefined threshold to a skin location with which an EMR-emitting end of the device is in contact.

28. The device of claim 22, wherein the safety system inhibits delivery of EMR exceeding a predefined threshold to the skin during a treatment session.

29. The device of claim 28, wherein a treatment session comprises a temporal period following activation of the device.

30. The device of claim 28, wherein the safety system comprise a controller tracking an amount of EMR energy being applied to a skin location, the controller inhibiting delivery of EMR to the skin upon the energy reaching the threshold.

31. The device of claim 28, wherein the controller is configured to de-activate the source to inhibit delivery of EMR to the skin.

32. The device of claim 7, wherein the scanner comprises at least one stepper motor.

33. The device of claim 1, wherein the EMR source generates EMR with one or more wavelengths in a range of about 300 nm to about 11,000 nm.

34. The device of claim 1, wherein the EMR source is a coherent light source.

35. The device of claim 1, wherein the EMR source is a single diode laser.

36. The device of claim 31, wherein the EMR source comprises a plurality of diode lasers.

37. The device of claim 1, wherein the light source is at least one diode laser bar.

38. The device of claim 1, wherein the light source is an incoherent light source.

39. The device of claim 38, wherein the incoherent light source can be selected from the group consisting of light emitting diodes (LED), arc lamps, flash lamps, fluorescent lamps, halogen lamps, and halide lamps.

40. The device of claim 1, wherein the housing comprises at least two separable modules one of which contains the EMR source and the other contains the EMR delivery mechanism.

41. The device of claim 40, wherein the modules include mating connectors for removably and replaceably engaging to one another.

42. The device of claim 40, further comprising a sensor system capable of sensing the type of EMR source and indicating the type to the scanner.

43. The device of claim 1, further comprising a cooling mechanism thermally coupled to the EMR source.

44. The device of claim 43, wherein the cooling mechanism comprises a thermoelectric cooler for extracting heat from the EMR source.

45. The device of claim 43, wherein the cooling mechanism comprises a thermal mass for extracting heat from the EMR source.

46. The device of claim 1, further comprising a rechargeable power supply disposed in the housing.

47. The device of claim 1, further comprising a docking station adapted for coupling to the housing, the docking station comprises circuitry for recharging the power supply.

48. The device of claim 1, wherein the EMR delivery path comprises a plurality of microlenses.

49. The device of claim 1, wherein discrete locations are contained within a skin portion requiring treatment.

50. The device of claim 1, further comprising a lotion dispenser coupled to the housing.

51. A photocosmetic system, comprising
a handheld portion extending from a proximal end to a distal end,
an EMR source disposed in the handheld portion,
a plurality of EMR-delivery modules, each of the modules being adapted for removable and replaceable coupling to the distal end of the handheld portion for delivery of light from the source to a plurality of distributed discrete skin locations,
wherein each of the light-delivery module provides a different pattern of the discrete locations.

52. The device of claim 51, wherein the handheld portion and the modules include mating connectors for removably and replaceably engaging to one another, such that a combination of the handheld portion and each module provides a handheld device.

53. The system of claim 51, wherein the patterns formed by the modules vary in area.

54. The system of claim 51, wherein the patterns formed by the modules vary in pitch.

55. The system of claim 51, wherein the patterns formed by the modules vary in shape.

56. The system of claim 51, wherein the patterns formed by the modules vary in focal depth.

57. The system of claim 51, wherein the proximal end is capable of being coupled to a docking station.

58. The system of claim 51, wherein the handheld portion further comprises a power source.

59. The system of claim 58, wherein the proximal end is capable of being coupled to a docking station, wherein the docking station comprises circuitry for recharging the power source.

60. A photocosmetic device, comprising
a housing extending from a proximal end to a distal end,
a plurality of light sources disposed in the housing configured to direct light through the distal end of the housing to a plurality of separated discrete skin locations,
a motion sensor mounted to the housing to sense a speed of movement of the distal portion to the skin,
a controller in communication with the motion sensor and the light sources, the controller controlling the sources based on the speed so as to direct light from the source to a plurality of separated discrete skin locations.

61. The photocosmetic device of claim 60, wherein the controller can control the selective activation of the sources.

62. The photocosmetic device of claim 60, wherein the sources are pulsed and the controller controls the repetition rate of the pulses.

63. (canceled)

64. A method for performing fractional treatments of tissue using a handheld photocosmetic device, comprising:
irradiating in a first treatment a plurality of separated treatment spots within a target area of tissue with EMR, wherein the total area of the plurality of treatment spots is less than the area of the target area;
irradiating in a second treatment a second plurality of separated treatment spots within the target area of tissue with EMR, wherein the total area of the second plurality of treatment spots is less than the area of the target area;
wherein the second irradiating step occurs after the first irradiating step and wherein at least the second irradiating step is performed using a self-contained handheld photocosmetic device.

65.-72. (canceled)
